(19) United States
(12) Patent Application Publication
(10) Pub. No.: US 2014/0088429 A1
(43) Pub. Date: Mar. 27, 2014

(54) ULTRASOUND IMAGING SYSTEM AND METHOD

(71) Applicants: Alexander Lomes, Moshav Husan (IL); Matityahu Shirizly, Modiln (IL)

(72) Inventors: Alexander Lomes, Moshav Husan (IL); Matityahu Shirizly, Modiln (IL)

(73) Assignee: Orcasonix Ltd., Natania (IL)

(21) Appl. No.: 14/119,911

(22) PCT Filed: May 24, 2012
(86) PCT No.: PCT/IB2012/052624

§ 371 Date: Nov. 25, 2013

Related U.S. Application Data
(60) Provisional application No. 61/489,737, filed on May 25, 2011.

(51) Int. Cl. A61B 8/08; A61B 8/00; A61B 8/14

(52) U.S. Cl. CPC A61B 8/5238; A61B 8/145; A61B 8/4461; A61B 8/4483
USPC 600/444; 600/443; 600/447; 600/445

(57) ABSTRACT

A method and system of producing an ultrasound image of an imaging region of a body, the image comprising pixels, the method comprising:
a) transmitting time-varying ultrasound into the imaging region, over a time interval, from a surface of the body, the transmitted ultrasound simultaneously having an angular spread in the imaging region corresponding to a plurality of the pixels of the image; and
b) receiving echoes of the transmitted ultrasound, and recording received signals of the echoes;
c) combining the received signals at the different sub-intervals of the time interval based on said time varying, according to expected ultrasound propagation times to scatterers localized at different pixels, to find image densities at the pixels.

### RELATED APPLICATION/S

This application claims the benefit of priority under 35 USC 119(e) of U.S. Provisional Patent Application No. 61/489,737 filed 25 May 2011, the contents of which are incorporated herein by reference in their entirety.

### FIELD AND BACKGROUND OF THE INVENTION

The present invention, in some embodiments thereof, relates to an ultrasound imaging system and, more particularly, but not exclusively, to a medical ultrasound imaging system.

Conventional ultrasound imaging systems are used extensively for medical imaging. Images of an imaging region are created by using a narrow beam of ultrasound pulses, and measuring the echo time in a given direction, for example from reflections from tissue boundaries. Then the direction of the beam is changed, either mechanically or using a phased array, and echo times are measured for the new direction, until a desired range of directions is covered. The beam is made as narrow as possible, in order to make the lateral resolution as fine as possible. Typically the beam width is diffraction limited, and the beam may be focused at a distance within the imaging region, sometimes using dynamic focusing.

Computerized tomographic methods have been used to reconstruct images from ultrasound, using profiles of absorption, acoustic velocity, and reflection with the Doppler effect. Absorption profiles were used by J. F. Greenleaf, S. A. Johnson, S. L Lee, G. T. Herman and E. H. Wood, “Algebraic reconstruction of spatial distribution of acoustic absorption within tissue from their two dimensional acoustic projections,” in Acoustical Holography, Vol. 5, P. S. Green Ed. New York: Plenum Press, 1974, pp. 591-603. Acoustic velocity profiles were used by J. F. Greenleaf, S. A. Johnson, W. F. Samayoa and F. A. Duck, “Algebraic reconstruction of spatial distribution of acoustic velocities in tissue from their time of flight profiles,” in Acoustical Holography, Vol. 6, N. Booth Ed. New York: Plenum Press, 1975, pp. 71-90. Reflection profiles, using the Doppler effect to distinguish moving targets, were used by G. Wade, S. Elliott, I. Khogeer, G. Flesher, J. Eisler, D. Mensa, N. S. Ramesh and G. Heidbreder, “Acoustic echo computer tomography,” in Acoustical Imaging, Vol. 8, A. F. Metherell Ed. New York: Plenum Press, 1978, pp. 565-576. Greenleaf and Ylitalo, “Doppler Tomography,” in 1986 Ultrasonics Symposium, IEEE, also used a Doppler tomographic method, to detect the existence of flowing fluid in a phantom. Ultrasonic imaging using tomography with the Doppler effect, caused by a linearly moving transducer, was described by K. Nagai and J. F. Greenleaf, “Ultrasonic imaging using the Doppler effect caused by a moving transducer” Optical Engineering, 1990, Vol. 29, pp. 1249-1254. The production of ultrasonic images by artificially moving the object while keeping the transducer static was described by Hai_Dong Liang et al [Hai_Dong Liang, Michael Halliwell, Peter N T Wells, “Doppler Ultrasonic Imaging”, in Acoustical Imaging, Vol 25, Edited by Michael Halliwell and Peter N. T. Wells, pp 279-288]. In all this work a backprojection algorithm, similar to those used in X-ray CT, was applied to process the Doppler shift frequencies from moving or rotating objects containing scattering targets, and continuous wave (CW) ultrasound was used.

U.S. Pat. No. 6,622,560 to Song et al describes using a pulse compression technique with a spread spectrum signal to improve range resolution in ultrasound imaging.

A review of synthetic aperture radar (SAR) is given by Berens, P. (2006) Introduction to Synthetic Aperture Radar (SAR). In Advanced Radar Signal and Data Processing (pp. 3-1-3-14). Educational Notes RTO-EN-SET-086, Paper 3. Neuilly-sur-Seine, France: RTO, available from: www.rto.nato.int/abstracts.asp. The idea of SAR is to transmit pulses and store the scene echoes along a synthetic aperture (i.e. the path of the SAR sensor) and to combine the echoes afterwards by the application of an appropriate focusing algorithm. The combination is carried out coherently.

Range migration correction algorithms in SAR are described in section 2.6.1.2.3 on “Range Doppler” of the ASAR Handbook published by the European Space Agency, downloaded on May 17, 2012 from www.envisat.esa.int/handbooks/asar/CNTR2-6-1-2-3.htm.

Additional background art includes L. J. Cutrona, “Synthetic Aperture Radar,” Chapter 21, in M. I. Skolnik, Radar Handbook, Third Edition, McGraw-Hill, 2008.

### SUMMARY OF THE INVENTION

An aspect of some embodiments of the invention concerns an ultrasound imaging system in which an image of an imaging region of a body is reconstructed from wide angle pulses of ultrasound, or modulated continuous ultrasound, transmitted and/or received at many different locations on the surface of the body.

There is thus provided, in accordance with an exemplary embodiment of the invention, a method of producing an ultrasound image of an imaging region of a body, the image comprising pixels, the method comprising:
a) transmitting time-varying ultrasound into the imaging region, over a time interval, from a surface of the body, the transmitted ultrasound simultaneously having an angular spread in the imaging region corresponding to a plurality of the pixels of the image; and
b) receiving echoes of the transmitted ultrasound, and recording received signals of the echoes;
   wherein one or both of the transmitting and the receiving is done at a different location during each of a plurality of different sub-intervals of the time interval; and
c) combining the received signals at the different sub-intervals of the time interval based on said time varying, according to expected ultrasound propagation times to scatterers localized at different pixels, to find image densities at the pixels.

Optionally, combining the received signals at the different sub-intervals comprises using a dependence of a phase of the received signals on sub-interval, due to a change in the location of the transmitting, the receiving, or both, in the different sub-intervals.

Optionally, combining the received signals at the different sub-intervals comprises finding a spectrum of at least the dependence of phase of the received signals on sub-interval.

Optionally, the method comprises using the spectrum to find a density of ultrasound scatterers as a function of azimuthal direction from the transducers, and range.

Optionally, the method comprises using the density of ultrasound scatterers as a function of azimuthal direction and range, to find a density of ultrasound scatterers as a function of azimuthal direction from the tranducers, and depth into the imaging region.

Optionally, combining the received signals at the different sub-intervals comprises matching the dependence of the phase of the received signals on sub-interval to an expected dependence of phase of the received signals on sub-interval, for scatterers located at different depths and lateral positions.

Optionally, the time-varying ultrasound within each sub-interval is frequency modulated.

Optionally, combining the received signals at the different sub-intervals comprises:
a) using a phase dependence of the received signal within sub-intervals to find a distribution of ranges of the echoes for the different sub-intervals; and
b) using phase differences between different sub-intervals, to find a distribution of azimuthal directions of the echoes for different ranges;
   wherein the phase changes within a sub-interval are at least 30 times as fast as the phase changes between sub-intervals.

Optionally, combining the received signals at the different sub-intervals comprises using differences in both amplitude and phase of the received signals at the different sub-intervals.

Optionally, combining the received signals at the different sub-intervals comprises using differences in phase of the received signals over a range of different locations of the transducers that is equal to at least half the maximum depth of the imaging region.

Optionally, the image densities at the pixels have a resolution in a direction lateral to the depth, that decreases by less than 30%, over at least one portion of the imaging region in which the depth increases by a factor of 2 or more.

Optionally, the image densities of the pixels have a resolution of better than 2 mm in a lateral direction, at a depth of at least 20 cm.

Optionally, the average frequency of the ultrasound is less than or equal to 2 MHz.

Optionally, the depth of at least 20 cm includes at least 10 cm of fat.

Optionally, transmitting is done with a transducer or transducer array that has a total width less than 2 mm

Optionally, the imaging region includes a portion within which the depth varies by more than a factor of 2, and the lateral width over which imaging data is acquired has an average lateral width that is more than 90% of the maximum lateral width in this portion of the imaging region.

Optionally, the image densities of the pixels have a spatial accuracy better than 2 mm in depth, at a depth of at least 20 cm.

Optionally, the image densities of the pixels have a spatial accuracy better than 2 mm laterally, at a depth of at least 20 cm.

Optionally, during each of the different sub-intervals, the transmitting is done at a same location as the receiving, or less than 10 mm from the receiving.

Optionally, the transmitting is done by a transmitter and the receiving is done by a receiver that move together to a different location at each of the different sub-intervals.

Optionally, the transmitter and receiver move substantially in a straight line or geodesic adjacent to the surface of the body.

Optionally, the transmitter and receiver move at a speed between 1 and 10 m/s.

Optionally, the transmitter and receiver move a total of at least 1 cm over the time interval.

Optionally, the transmitter and receiver move a total of at least 10 wavelengths of ultrasound waves of an average frequency of the transmitted ultrasound waves.

In an embodiment of the invention, the transmitter and receiver move substantially in a circular arc with a direction of curvature parallel to the surface of the body.

Optionally, the transmitter and receiver move a total of at least 5 cm over the time interval.

Optionally, transmitting and receiving is done for a plurality of different time intervals, each for different imaging regions, in different angular ranges of the motion of the transmitter and receiver in the circular arc, and combining the received signals to form image densities is done for each of said time intervals and imaging regions.

Optionally, the transmitter and receiver move at substantially constant speed during the time interval.

Optionally, the method also comprises:
a) measuring deviations of the motion of the transmitter and receiver from constant speed; and
b) adjusting the expected ultrasound propagation times according to the measured deviations.

Optionally, transmitting time-varying ultrasound comprises transmitting pulses of ultrasound, at least one pulse in each of the sub-intervals.

Optionally, transmitting is done by a transmitter that moves to a different location in different sub-intervals, and transmits a pulse at least every 5 mm of its motion.

Optionally, at least one of the pulses is frequency modulated, with pulse length times frequency bandwidth greater than 3.

Alternatively or additionally, at least one of the pulses has pulse length times frequency bandwidth less than 3.

Optionally, at least one of the pulses has a frequency bandwidth greater than 0.5 MHz.

Optionally, transmitting the pulses comprises spacing the pulses apart by a time greater than twice a sound speed transit time of a greatest distance across the imaging region.

Optionally, combining the received signals is done also according to expected dispersion in body tissue, losses in body tissue, or both.

In an embodiment of the invention, transmitting and receiving are done by a separate transducer at each of the locations, and a different transducer is used for transmitting ultrasound in different sub-intervals, or a different transducer is used for receiving ultrasound in different sub-intervals, or both.

Optionally, the imaging region is comprised in a slice extending into the body from said surface, narrower in a direction across the slice on the surface than in a direction along the slice on the surface, and the transducers used for transmitting are arranged substantially in a straight line along the slice on or adjacent to the surface, and the transducers used for receiving are arranged substantially in a straight line along the slice on or adjacent to the surface.

Optionally, at least one of the transducers is used for transmitting in one sub-interval and for receiving in a different sub-interval.

Optionally, at least some of the transducers are arranged substantially in a row, and at least 80% of the transducers in the row are used for transmitting in different sub-intervals, and at least 80% of the transducers in the row are used for receiving in different sub-intervals.

Optionally, the imaging region is comprised in a slice extending into the body from said surface, the transmitting and receiving locations being on or adjacent to a portion of the surface that the slice extends from, the portion being narrower in a direction across the slice than in a direction along the slice.

Optionally, the transmitting is done by a transmitter and the receiving is done by a receiver, the same as or different from the transmitter, that move together in a direction substantially along the slice to a different location at each of the different sub-intervals.

Alternatively, the transmitting is done by a transmitter and the receiving is done by a receiver, the same as or different from the transmitter, that move together in a circular motion tangent to a direction substantially along the slice, to a different location at each of the different sub-intervals.

Optionally, transmitting ultrasound comprises transmitting over a narrower range of angles transverse to the slice, than along the slice.

Optionally, the receiving is done by a receiver comprising an array of receiving elements oriented in a direction transverse to the slice, and receiving an echo of the ultrasound comprises using phase differences between the signal received by different receiving elements to exclude components of the echo coming from directions too far from directions parallel to the slice.

Optionally, the transmitting is done by a transmitter comprising an array of transmitting elements oriented in a direction transverse to the slice, and transmitting the ultrasound comprises using phase differences between the different transmitting elements so that the transmitted ultrasound is substantially confined to directions of propagation that go through the slice.

Optionally, transmitting comprises transmitting with an angular distribution of power, parallel to the slice, at least 20 degrees wide, full width half maximum.

Optionally, receiving comprises receiving with an angular sensitivity, parallel to the slice, at least 20 degrees wide, full width half maximum.

Optionally, the image comprises a two-dimensional image.

There is further provided, according to an exemplary embodiment of the invention, a method of producing a three-dimensional ultrasound image, comprising repeatedly producing a two-dimensional image according to the method of an embodiment of the invention, for a plurality of different substantially parallel slices of an imaging volume of a body, and combining the two-dimensional images to form a three-dimensional image of the imaging volume.

Optionally, a lateral resolution of the image is better than one wavelength of an average frequency of the transmitted ultrasound, down to a depth of at least 20 such wavelengths beneath the surface.

Optionally, combining takes into account differences in the phase of the expected received signal for different sub-intervals.

There is further provided, according to an exemplary embodiment of the invention, a system for calculating density values of pixels of an ultrasound image of an imaging region of a body, comprising:
a) one or more transmitting transducers adapted to transmit time-varying ultrasound waves into the imaging region with an angular spread of at least 20 degrees, when placed on or adjacent to a surface of the body at any of a plurality of different transmitting locations;
b) one or more receiving transducers, the same as or different from the transmitting transducers, adapted to receive an echo of the transmitted ultrasound waves from the imaging region, when placed on or adjacent to the surface at any of a plurality of different receiving locations, and to generate a received signal thereof;
c) a location changing device adapted to change the transmitting location, or adapted to change the receiving location, or adapted to change both; and
d) a controller adapted to calculate the image density value of different pixels, by combining the received signals according to expected ultrasound propagation times to and from the pixels, for a plurality of different transmitting locations, a plurality of different receiving locations, or both.

Optionally, the location changing device is adapted to move a transmitting transducer, a receiving transducer, or both, from one of the locations to another.

Optionally, the system comprises a movement sensor that senses the location as a function of time of at least one transducer that is moved by the location changing device, and generates data thereof, wherein the controller is adapted to use said data to adjust the expected ultrasound propagation times.

In an exemplary embodiment of the invention, the location changing device is adapted to move the transducer substantially in a straight line or geodesic along or adjacent to said surface of the body.

Optionally, the location changing device comprises:
a) a rigid acoustically transparent plate, a lower surface of which is adapted to remain in good acoustic contact with the surface of the body, over a range of the locations; and
b) a motor adapted to move the transmitting transducer, receiving transducer, or both, along an upper surface of the plate, opposite the lower surface, over the range of the locations, while the transducer remains facing the plate, in direct or indirect contact with the upper surface.

Optionally, the location changing device also comprises a rolling element surrounding the transducer, with a lower friction inner surface in contact with the transducer and a higher friction outer surface in contact with the plate, adapted to roll along the upper surface of the plate without slipping when the motor moves the transducer, while the transducer slides against the inner surface so as to remain in an orientation facing the plate, the rolling element providing good acoustic contact between the transducer and the plate as the transducer moves.

Alternatively, the location changing device comprises a motor adapted to move the transducer substantially in a circular arc.

Optionally, the location changing device comprises a rigid circular rotating portion on which the transducer is mounted, and a motor that causes the rotating portion to rotate, and the system also comprises a stationary portion that remains fixed when the rotating portion rotates.

Optionally, both a transmitting transducer and a receiving transducer, the same as or different from the transmitting transducer, are mounted on the rotating portion, and the rotating portion also comprises drive circuitry for driving the transmitting transducer and receiving circuitry for generating a data signal from the receiving transducer.

Optionally, the circular portion is more than 4 cm in diameter.

Optionally, the stationary portion comprises a circular rigid acoustically transparent case adapted to press against the surface of the body, the transducer being located inside the case and moving around the inside of the case in a circular path when the rotating portion rotates, the case containing fluid that acoustically couples the case to the moving transducer.

Optionally, the system comprises a wireless interface between the rotating and stationary portions.

In an exemplary embodiment of the invention, the one or more transmitting transducers comprise an array of transmitting transducers, the one or more receiving transducers comprise an array of receiving transducers, or both, and the location changing device comprises switching circuitry that switches which transducer in the array of transmitting transducers is used for transmitting, or which transducer in the array of receiving transducers is used for receiving, or both.

Optionally, the system comprises transmission circuitry that generates a waveform for the transmitted ultrasound waves, and receiving circuitry that generates the received signal from the echo, wherein the switching circuitry successively connects and disconnects different transducers in the array of transmitting transducers to the transmission circuitry, or successively connects and disconnects different transducers in the array of receiving transducers to the receiving circuitry, or both.

Optionally, the array of transmitting transducers, the array or receiving transducers, or both, extends over a distance more than 1 cm, with different locations of the transducers spaced at intervals no greater than 5 mm

Optionally, the system comprises signal generating circuitry that drives the transmitting transducers to transmit such a waveform and frequency of ultrasound, and with such a range and spacing of the locations, and with the controller programmed to calculate the image densities of the pixels in such a manner, that the image has a resolution better than 2 mm down to depth of at least 20 cm.

Optionally, the system comprises signal generating circuitry that drives the transmitting transducers to transmit such a waveform and frequency of ultrasound, and with such a range and spacing of the locations, and with the controller programmed to calculate the image densities of the pixels in such a manner, that the image has a resolution better than an average wavelength of the transmitted ultrasound to depth of at least 20 such wavelengths.

There is further provided, in accordance with an exemplary embodiment of the invention, a method of forming an image of an imaging region in a body comprising:
a) transmitting ultrasound waves into the imaging region;
b) receiving echoes of the transmitted ultrasound, from the imaging region; and
c) reconstructing an image of the imaging region from data of the received echoes;
   wherein the image has a resolution in a direction lateral to depth into the body, that decreases by less than 30%, over at least one portion of the imaging region in which the depth increases by a factor of 2 or more.

There is further provided, in accordance with an exemplary embodiment of the invention, a system for reconstructing ultrasound images of an imaging region in a body, comprising:

a) an acoustically transparent window adapted for being placed in good acoustic contact with the body, in the form of a circular path;

b) a transmitting transducer and a receiving transducer that is the same as or different from the transmitting transducer, adapted to transmit ultrasound through any part of the acoustically transparent window and into the imaging region of the body, and to receive echoes of the transmitted ultrasound from the imaging region;

c) a rotary motor that drives the transmitting transducer and receiving transducer in a circular path along the acoustically transparent window, while they are transmitting and receiving data;

d) receiving circuitry adapted to generate a data signal of received echoes from the receiving transducer; and

e) a controller adapted to reconstruct an image of the imaging region from the data signal.

There is further provided, in accordance with an exemplary embodiment of the invention, a method of producing an ultrasound image of an imaging region of a body, the image comprising pixels, the method comprising:
a) transmitting time-varying ultrasound into the imaging region, over a time interval, from a surface of the body, the transmitted ultrasound simultaneously having an angular spread in the imaging region corresponding to a plurality of the pixels of the image; and
b) receiving echoes of the transmitted ultrasound, and recording received signals of the echoes;
   wherein one or both of the transmitting and the receiving is done at a plurality of different locations; and
c) combining the received signals based on said time varying, according to expected ultrasound propagation times to scatterers localized at different pixels, to find image densities at the pixels.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic side cross-sectional view of a linearly moving ultrasound imaging system, according to an exemplary embodiment of the invention;

FIG. 2A is a schematic side cross-sectional view of the system in FIG. 1, showing distances traveled by different ultrasound pulses to different scatterers;

FIG. 2B is a schematic side cross-sectional view of the system in FIG. 1, showing an increase in ultrasound illumination time of a given location, with increasing depth;

FIG. 3A is a flowchart of a method of reconstructing an ultrasound image, according to an exemplary embodiment of the invention;

FIG. 3B schematically shows plots of a frequency modulated pulse, before and after filtering with a fast reference function, as well as the frequency spectrum of a fast and slow reference function, according to an exemplary embodiment of the invention;

FIG. 4 is a schematic perspective view of an ultrasound imaging slice and a transducer array, according to an exemplary embodiment of the invention;

FIG. 5 is a schematic perspective view of a system for ultrasound imaging with a linearly moving transducer, according to an exemplary embodiment of the invention;

FIG. 6 is a schematic cross-sectional view of imaging slices used by an ultrasound transducer, according to an exemplary embodiment of the invention;

FIG. 7 is a schematic side cross-sectional view of a system for ultrasound imaging with a linearly moving transducer, according to another exemplary embodiment of the invention;

FIG. 8A is a block diagram showing modules of a controller used for an ultrasound imaging system, according to an exemplary embodiment of the invention;

FIG. 8B is a flowchart for control software used to obtain an image, for the system shown in FIG. 5;

FIG. 9 is a schematic perspective view of an ultrasound imaging system with a circularly moving transducer, according to an exemplary embodiment of the invention;

FIG. 10A is a block diagram of elements of the system shown in FIG. 9;

FIG. 10B is a flowchart for control software used to obtain an image, for the system shown in FIG. 9;

FIG. 11 is a schematic side cross-sectional view of the system shown in FIG. 9;

FIG. 12 is schematic perspective view and block diagram of a pseudo-motion ultrasound imaging system according to an exemplary embodiment of the invention;

FIG. 13 shows two ultrasound images of a phantom, one from a conventional ultrasound imaging system and one from a system similar to the system of FIG. 12;

FIG. 14 shows two ultrasound images of a phantom, one from a conventional ultrasound imaging system and one from a system similar to the system of FIG. 12; and

FIG. 15 schematically shows a frequency spectrum of a transmitted ultrasound pulse and a received ultrasound pulse.

### DESCRIPTION OF SPECIFIC EMBODIMENTS OF THE INVENTION

The present invention, in some embodiments thereof, relates to an ultrasound imaging system and, more particularly, but not exclusively, to a medical ultrasound system.

An aspect of some embodiments of the invention concerns a method and system for ultrasound imaging of a region of a body, in which many ultrasound pulses, or modulated continuous waves, are sequentially transmitted into the region, with a broad angular spread, and echoes are received, with the transmitter, the receiver, or both, located at different places adjacent to the surface of the body, for different pulses. The received data of the echo signals for different pulses is used to calculate an image of the region, based on time-delayed transmitted signals that would be expected from ultrasound scatterers at different locations in the imaging region, and hence at different distances from the transmitter and receiver, the distances also changing from one pulse to the next. The pulses may be frequency modulated, and the method may include finding a distribution of echoes over range, for each pulse, using fast matched filtering of the individual pulses. The method may include finding the distribution of echoes over azimuthal direction for each range, by performing a spectral analysis over different pulses, of the data for each range, for example finding a distribution over Doppler shift for each range. The method may include converting a distribution of echoes over azimuthal direction and range, to a distribution over azimuthal direction and depth below the surface, for example using a range migration correction algorithm, or a backprojection algorithm. The method may include applying a filter, such as a slow frequency modulated filter over different pulses, to find the echo distribution as a function of lateral position and depth, from the distribution as a function of depth and azimuthal direction, thereby reconstructing a two-dimensional image of the region.

Optionally, the method comprises using complex numbers representing both phase and amplitude of received ultrasound echo signals. Optionally, phase differences on a fast time scale, in a frequency modulated pulse, are used to find a distribution of echoes over range, and phase differences on a slow time scale, due to Doppler shifts that depend on an azimuthal angle with respect to a direction of motion or pseudo-motion of a transducer, are used to find a distribution of echoes over lateral position, for a given depth.

Optionally, a transmitting and/or receiving ultrasound transducer moves to a different location adjacent to the surface of the body, for each new pulse, using an acoustically transparent plate that is in contact with the surface of the body, and a motor that moves the transmitter and receiver (which may be the same transducer) over the surface of the plate, transmitting and receiving ultrasound pulses at different locations. Optionally the motor moves the transducers in a straight line. Alternatively, the motor is a rotary motor that moves the transducers in a circle.

Alternatively, instead of moving the transducers over the surface of the body while transmitting and receiving ultrasound pulse, the system comprises a static array of transducers in acoustic contact with the surface of the body, and transducers in different locations on the array sequentially transmit and receive ultrasound pulses.

Optionally, the region that is imaged comprises a thin slice, and the image is a two-dimensional image. Optionally, several such slices, parallel to each other, are imaged one after the other to produce a three-dimensional image.

Optionally, the imaging method produces images with a lateral resolution that is relatively independent of depth within the imaging region. For example, the lateral resolution is better than 2 mm, or better than 1 mm, or better than 0.5 mm, or better than two wavelengths of the average frequency of transmitted ultrasound, or better than 1 wavelength, or better than half a wavelength, down to a depth of 10 cm, or down to a depth of 20 cm, or down to a depth of 30 cm, or down to a depth of 20 wavelengths, or 50 wavelengths, or 100 wavelengths, or 200 wavelengths. As used herein, resolution refers to the minimum distance at which two lines, of maximum contrast with the background, can be separated. Alternatively, the resolutions given here may refer to the minimum distance at which two points of maximum contrast can be separated.

Optionally, the imaging method measures depth and lateral position absolutely.

An aspect of some embodiments of the invention concerns an ultrasound imaging system, in which there is transmitter and receiver, using the same or different transducers, mounted on a spinning rotor, causing the transmitter and receiver to follow a circular path adjacent to a surface of the body. Ultrasound waves are transmitted into an imaging region of a body by the transmitter, and echoes are detected by the receiver, which generates receiver data that is used to reconstruct an image of the imaging region.

An aspect of some embodiments of the invention concerns a method and system for ultrasound imaging of a region of a body, in which ultrasound is transmitted into the region, with a broad angular spread, and echoes are received, with the transmitter, the receiver, or both, located at different places adjacent to the surface of the body, at different times, or simultaneously using multiple transmitters and/or receivers. The received data of the echo signals for different locations of the transmitter and/or receiver is used to calculate an image of the region, based on time-delayed transmitted signals that would be expected from ultrasound scatterers at different locations in the imaging region, and hence at different distances from the transmitter and receiver, the distances also changing according to the position of the transmitter and/or receiver.

An aspect of some embodiments of the invention concerns an ultrasound imaging system using synthetic aperture imaging methods.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Linearly Moving System

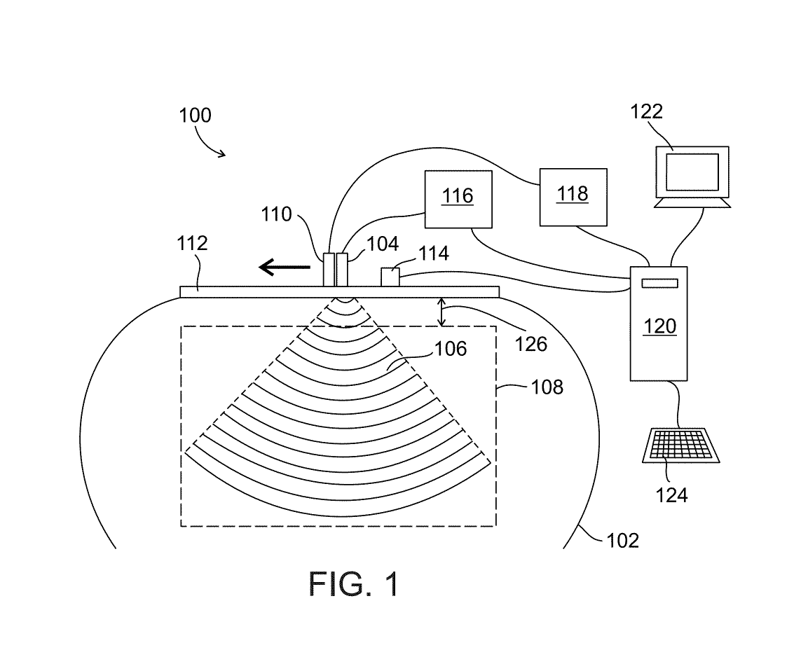

Referring now to the drawings, FIG. 1A illustrates a system 100 for producing an ultrasound image of a body 102. A transmitting ultrasound transducer 104 emits a relatively wide angle beam of ultrasound waves 106, into an imaging region 108 of body 102, for example a thin slice in the plane of the drawing. The beam is, for example, at least 10 degrees wide, full width at half maximum intensity, or at least 20 degrees wide, or at least 30 degrees wide, or at least 45 degrees wide, or at least 60 degrees wide, or at least 90 degrees wide, or at least as wide as 5 times the lateral resolution of the image everywhere in the imaging region, or at least 10 times, or at least 20 times, or at least 50 times, or at least 100 times. These numbers refer to the width of the beam within the plane of the slice; perpendicular to the plane of the slice, the angular spread of the beam may be quite narrow. A receiving ultrasound transducer 110 detects echoes of ultrasound waves 106 from scatterers in imaging region 108, for example at boundaries between tissues with different acoustic impedance. One or both of transmitting transducer 104 and receiving transducer 110 move along a surface of body 102, optionally together, transmitting and receiving ultrasound at a plurality of different locations. Optionally, the transmitting and receiving transducer are always within 10 mm or each other, or within 5 mm of each other, or within 2 mm of each other, or within 1 mm of each other. In some embodiments of the invention, the same transducer is used both for transmitting and receiving, for example by switching the transducer to a receiving mode after finishing transmitting a pulse of ultrasound.

In some embodiments of the invention, the transducers are not directly in contact with the body, but move along a rigid, acoustically transparent plate 112, and the ultrasound waves travel from the transmitting transducer into the body through the plate, and the echoes travel from the body into the receiving transducer through the plate. This has the potential advantages that the surface of the body is forced to maintain a certain shape, for example a flat plane if plate 112 is flat, making it possible to better control and determine the location of the transducers as they are moved across the body, and the motion of the transducers may be less likely to change the configuration of tissue inside the body as they move, thereby avoiding motion artifacts in the image. Such a plate also makes it possible to reduce drag on the transducers, and prevents damage to the body by the transducers even if they are moving at relatively high speed, for example at 1 to 10 meters per second.

A motor, not shown in FIG. 1, is optionally employed to move the transducers in a well controlled way, as will be described below. Any irregularity in the motion is optionally measured by a position or motion sensor 114, and taken into account when reconstructing the image.

Drive circuitry 116, controlling transmitting transducer 104, produces ultrasound waves of a desired waveform, for example short pulses, or “chirped” (frequency modulated) pulses. Receiver circuitry 118, attached to receiving transducer 110, converts receiver data into a received signal, for example a digital signal. A controller 120, for example a personal computer or a specialized processor, optionally stores the receiver signal, and uses it to reconstruct an image of imaging region 108, for example a two-dimensional image in the case of an imaging region that is a thin slice. Controller 120 also optionally uses data about the positions of the transmitting and receiving transducers as a function of time, from sensor 114, in reconstructing the image, and may use other data as well. Controller 120 also optionally communicates with drive circuitry 116, to control and/or to sense the timing of the transmitted ultrasound pulses, and/or their waveform. A display device 122, such as a computer monitor or a printer, optionally displays the image for a user. A user interface 124, comprising for example a keyboard, a mouse and/or other input device, optionally allows a user to control one or more parameters of system 100.

In some embodiments of the invention, instead of using separated ultrasound pulses, frequency modulated continuous wave ultrasound is used. Using separated pulses has the potential advantage that there are intervals between pulses when the transmitting transducer is not operating, and it is possible to operate the receiver only during those intervals, when there is no danger of damage to the receiver circuitry by leakage of power from the transmitting transducer, or by ultrasound waves travelling only a short distance from the transmitting transducer to the receiving transducer. Nevertheless, it is possible to use frequency modulated continuous wave ultrasound, for example frequency modulated pulses with no break between them, using separate transmitting and receiving transducers, and not having the receiving transducer too close to the transmitting transducer. In the description that follows, separate ultrasound pulses are generally assumed, but it should be understood that frequency modulated continuous wave ultrasound can be used instead, in much the same way.

Although the embodiments of the invention described herein mostly concern medical imaging, in which the body is a human or animal body, it should be understood that the method can also be used with inanimate bodies, for example with equipment that is being inspected with ultrasound for non-destructive testing.

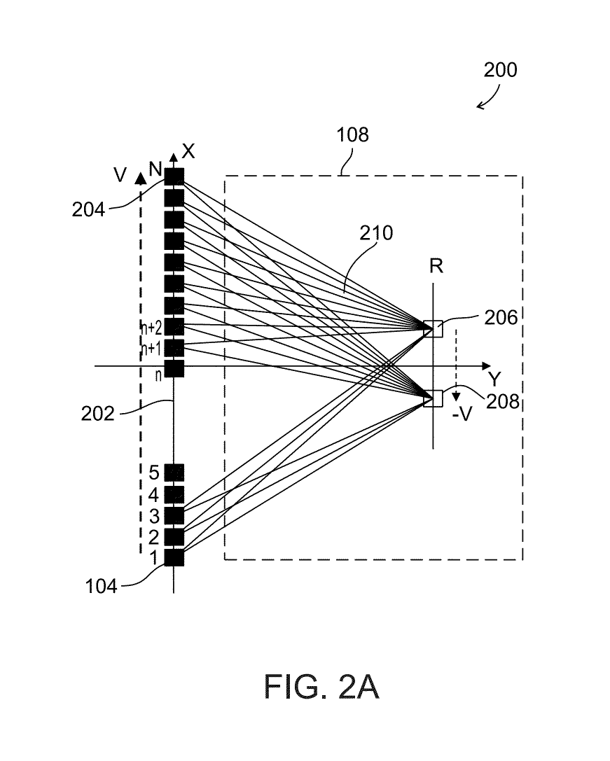

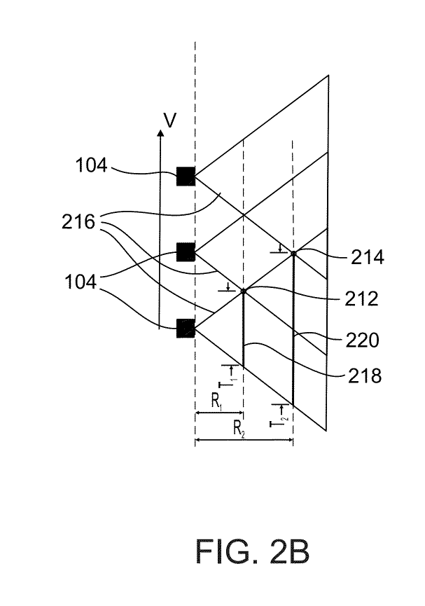

FIG. 2A illustrates how the changing distance from the transmitting transducer to a scatterer in the imaging region, and back to the receiving transducer, affects the received signal, and how this can be used to distinguish components of the received signal coming from scatterers at different lateral positions. Transducer 104 follows a path 202 in the x-direction, parallel to the surface of the body (shown vertically in FIG. 2A, rather than horizontally as in FIG. 1). As transducer 104 moves at a speed V, it transmits N ultrasound pulses, each pulse, labeled 1, 2, 3, 4, 5, . . . n, n+1, n+2, . . . N in FIG. 2A, transmitted from a different position. The transducer ends at position 204, the position at which the last pulse, pulse N, is transmitted. It is assumed in FIG. 2A that each pulse is received at approximately the same location where it was transmitted, neglecting the change in position of the transducer during the echo time of one pulse, and neglecting any difference in position between the transducer and receiver, if the same transducer is not used for both. The pulses are transmitted from locations in x that differ by Δx, at times that differ by Δx/V. Scatterers are shown localized at each of two points 206 and 208, in imaging region 108, at the same depth from transducer path 202. For each pulse n, the distance traveled by a pulse, from the transducer to the scatterer and back to the receiver, may in general be different, and this results in a phase difference between the received pulses. In particular, the phase may change by 4π sin θ Δx/λ from one pulse to the next, where λ is the wavelength of the ultrasound, at the center frequency of the transmitted pulse, and θ is the angle between the y-direction, and the direction of the path 210 between the transducer and the scatterer travelled by that pulse. Considering the signal of all of the received pulses in order as one long signal, and assuming the transmitted pulses are all in phase, the received signal will have a change in phase (expressed in radians) of 4π sin θ Δx/λ over the time Δx/V between pulses, and this may appear as a Doppler shift (expressed in cycles per second) of 2 sin θ V/λ in the frequency of the received signal. This is what one would expect, since the scatterer is moving at a velocity V in the −x direction relative to the transducer, and the component of the velocity, in the direction between the transducer and the scatterer, is V sin θ. The Doppler shift for the received signal coming from the scatterer at location 208 will generally be different from the Doppler shift coming from the scatterer at location 206, because the angle of the path between the transducer and the scatterer, relative to the y-axis, is different for location 206 and location 208. Hence the components of the received signal scattering from the locations 206 and 208 can be distinguished from each other, by their different Doppler shifts. If two scatterers are separated by a distance δ in x, and they are at the same depth y, then their angle to the transducer, at any given time, may differ by about δ/y, and their Doppler shift may differ by about 2Vδ/λy. It may be possible to resolve the two scatterers if their difference in Doppler shift, times the effective duration of time during which they are scattering ultrasound from the transducer, is greater than 1.

As shown in FIG. 2B, a given scatterer only contributes significantly to the received signal of the transducer, for a part of the entire time between pulse 1 and pulse N. The time depends on the depth of the scatterer. FIG. 2B shows a transducer 104 moving at a velocity V in the x-direction. The position of transducer 104 is shown at three different times during its motion. A scatterer 212 is located at a depth R₁, and a scatterer 214 is located at a greater depth R₂. Because of the finite angular spread of an ultrasound beam 216 transmitted by transducer 104, scatterer 212 only receives pulses from the transducer during a period 218 of duration T₁, and scatterer 214 only receives pulses from the transducer during a period 220 of duration T₂, which is greater than T₁. In fact, at least for depths that are less than the path length of the transducer, we expect that the width of the beam will be about equal to the depth y, so the duration during which the scatterer is contributing to the received signal will be about y/V. This would be true if the beam had an angular width of about 90 degrees. Even if the beam had an angular width greater than 90 degrees, the effective duration might still be about y/V, since at times outside this period, the distance between the transducer and the scatterer will be significantly greater, leading to greater attenuation of the echo, which may make much less of a contribution to the received signal than other scatterers that are not so far away from the transducer. From this expression for the duration, we find that the Doppler shift between two scatterers will make it possible to distinguish them if they are separated in the x-direction by a distance δ>λ/2, independent of depth y. This estimate ignores the finite width in x of the transducer, and if that is taken into account, the resolution in x may be about half the width of the transducer, still independent of depth y.

As noted above, the image reconstruction method uses Doppler shift, which depends on the relative speed of the scatterer to the moving transducer, to distinguish echoes from scatterers located at different positions. The image reconstruction method assumes that the scatterers in the imaging region are not moving, relative to the body, during the time ultrasound echo data is acquired. If ultrasound is reflected from a target that is moving, for example blood inside a blood vessel, then that target may appear displaced from its true position in the reconstructed image. Optionally, the operating parameters of the system are chosen, based on an expected speed of motion of different components in the imaging region, to keep such motion artifacts tolerably small. For example, the maximum echo time is kept small enough, and the points from which the ultrasound pulses are transmitted are kept far enough apart, so that the transducer can move much more quickly than any component of the image, even though this may require using relatively low frequency ultrasound, resulting in a lower resolution in the image, thereby reducing the resolution of the image to avoid motion artifacts in the image. Optionally, a desired tradeoff is found, between image resolution and motion artifacts. In some embodiments of the invention, motion artifacts are used to measure the speed of components of the image. For example if it is known where a blood vessel is actually located, then measuring the displacement of its apparent position in the image from its real image will provide information on the speed of the blood flowing through it.

Optionally, transducer 104 transmits a pulse of ultrasound, then waits until there has been time for an echo to reach receiving transducer 110 from the most distant part of imaging region 108, before emitting another pulse. This has the potential advantage that it is possible to immediately tell which pulse a given part of the received signal belongs to, making it potentially easier to analyze the received signals to reconstruct an image.

Optionally, transducer 110 only records received data after transducer 104 has finished transmitting a pulse. This is particularly important if the same transducer is used for transmitting and receiving, since the receiving circuitry could be damaged if it is connected to the transducer while the transducer is transmitting. Even if separate transducers are used for transmitting and receiving, the receiving circuitry may be damaged if the receiving transducer is very close to the transmitting transducer while it is transmitting. This results in a dead space 126, near the top of imaging region 108 in FIG. 1A, which is not imaged. The dead space optionally includes plate 112, which has the potential advantage that the receiver circuitry will not be exposed to the strong reflection that may be generated by the interface between plate 112 and body 102. As used herein, the imaging region is part of a slice that extends from the path of the transducers on the surface, into the body, but the imaging region only takes up part of the slice, because the dead region is not considered part of the imaging region.

Information on the length of a path, from a transmitter to a scatterer and back to a receiver, is optionally obtained in two different ways. The echo time of a pulse of ultrasound can be measured, either directly from the time delay, in the case of a short pulse, or using matching filters, in the case of a frequency modulated pulse. In addition, there is a phase change in ultrasound received at the receiving transducer, relative to the transmitted ultrasound, that depends on the path distance that it traveled. The echo time and the phase change can give complementary information, with the echo time often providing better depth resolution, and the phase change often providing better lateral resolution, as will be explained below.

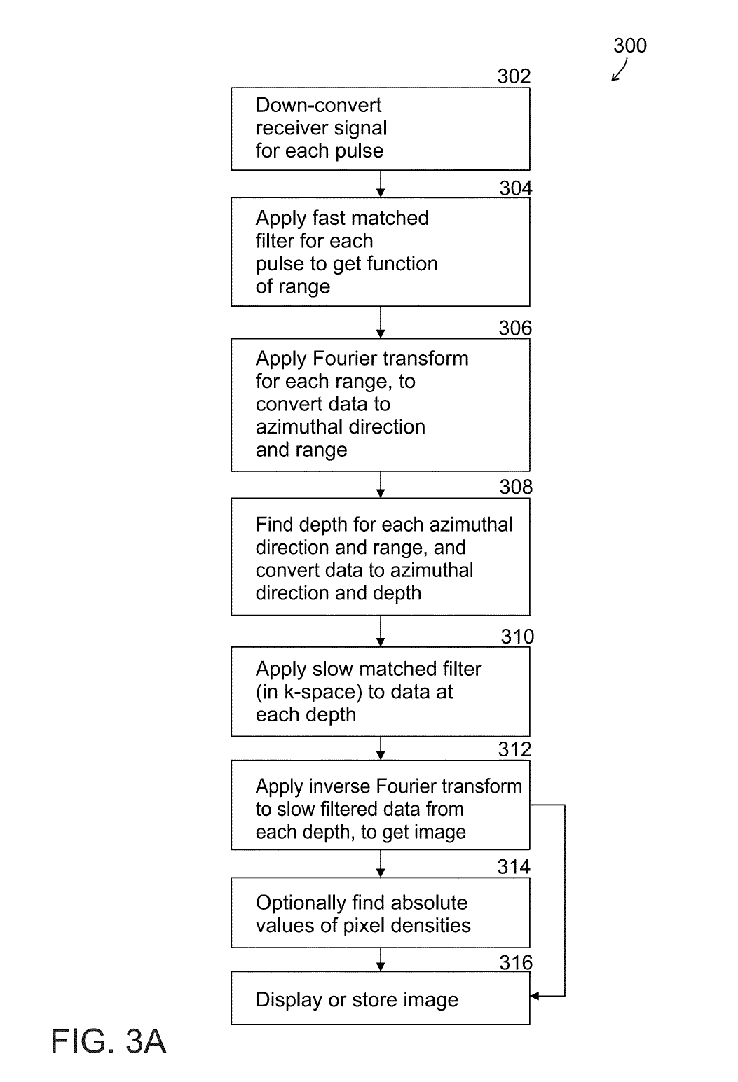

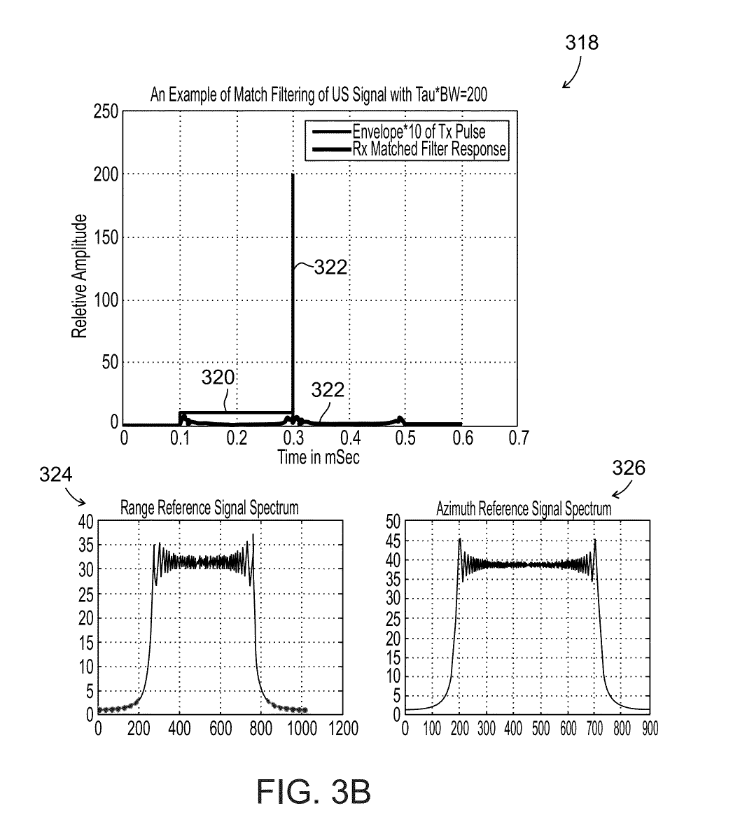

FIG. 3A shows a flowchart 300, for a method of reconstructing an image from received signals for each pulse. For illustrative purposes, the transmitted pulse of pulse number n is assumed to be a frequency modulated pulse, for example a linear frequency modulated pulse with a square envelope, of the form

$$\begin{matrix}{s}_{n}^{t} ( t ) = exp [ 2 π i {F}_{c} ( t - {t}_{n} ) + π i \frac{{B}_{0}}{{T}_{p}} {( t - {t}_{n} )}^{2} ] for \vert t - {t}_{n} \vert < \frac{1}{2} {T}_{p} \\ = 0 otherwise\end{matrix}$$

where tₙ is the time of the center of the nth pulse, for example tₙ=nT, where T is the time from one pulse to the next, F_{c} is the center frequency of the pulse, B₀ is the bandwidth, and Tₚ is the pulse length. Alternatively any other type of frequency modulated pulse is used, including various types of nonlinear frequency modulation known in the art. The envelope shape need not be the square envelope of width Tₚ given above, and other envelope shapes may be better for minimizing side lobes, and/or may be easier to produce; any envelope shape is optionally used. Typically B₀ is comparable to, but somewhat less than F_{c}, for example about 60% of 80% of F_{c}, since it may be difficult to make an ultrasound transducer with a greater bandwidth than that. F_{c} may be any ultrasound frequency used for medical ultrasound imaging, for example 0.5 MHz, 1 MHz, 1.5 MHz, 2 MHz, 3 MHz, 5 MHz, 10 MHz, or smaller, greater, or intermediate frequencies. The inventors have found that frequencies in the range of 1 to 2 MHz are particularly useful for many medical imaging applications, because they give fairly high resolution, for example 0.5 mm, 1 mm, or 2 mm, or larger, smaller, or intermediate values, and penetrate fairly far into tissue, for example up to 10 cm, 20 cm, or 30 cm, allowing an imaging region of that depth. For applications requiring a higher resolution and not requiring such a deep imaging region, higher frequencies are potentially advantageous. Optionally, B₀Tₚ is much greater than 1, for example up to 5, greater than 5, greater than 10, greater than 20, greater than 50, greater than 100, or greater than 200, or an intermediate number. Bandwidth and pulse length are defined here according to any standard definition known in the art, for example full width at half maximum. Having a frequency modulated pulse with large B₀Tₚ has the potential advantage that matching filters, as will be described below, can be used to find the echo time very precisely, to within a time of about 1/B₀, but the energy of the pulse is spread out over a much longer time Tₚ, allowing the power of the transmitting transducer and its electronics to be much lower, for a given total pulse energy, which determines signal to noise ratio. Using frequency modulated pulses with large B₀Tₚ also makes the system less sensitive to dispersion, than if short pulses were used. But short pulses with B₀Tₚ about 1 are also optionally used, for example with B₀Tₚ between 2 and 5, or between 1.5 and 2, or between 1.2 and 1.5, or between 1 and 1.2. Bandwidth and pulse length are defined here in such a way, for example, that B₀Tₚ cannot be less than 1.

The image densities are calculated from the received signals using any matching filters, that find the contribution to the signal of scattering from each location (x, y), by comparing the signal to signals that would be expected if there were a single scatterer localized at (x, y). For example, optionally the image density D(x, y) is found from

D(x,y)=∫dts_{ref}(t,x,y)sᵣ(t)

where s_{ref}(t, x, y) is the complex conjugate of the reflected signal that would be expected from a scatterer localized at (x, y), taking into account the time delays and Doppler shifts associated with an ultrasound wave travelling from the moving transmitting transducer, to the point (x, y), and returning to the receiving transducer. Here sᵣ(t) is the complete received signal for all the pulses, and the integral is over the time interval that the echoes for all the pulses are received. Although this algorithm is conceptually simple, the inventors have found that the somewhat more complicated method described below works better, producing an image efficiently with relatively low noise and high resolution.

At 302, the received signal is optionally down-converted to a signal Sₙ^{r}(t), removing the factor of exp(2πiF_{c}t), but keeping amplitude and phase information as a function of time. Optionally, this is done in real time when the received signal is generated, and only the down-converted signal is stored, which has the potential advantages of reducing storage requirements, and simplifying the calculation of the image. At 304, the received signal for each pulse n is filtered by convolving it with a fast reference function, with a time delay that would occur if it reflects from a scatterer localized at a distance R from the transducers. The fast reference function is the transmitted pulse for pulse n, given above. To find its time delayed form, note that the distance from the transmitter to a scatterer at a point (x. y), as a function of time t, is given by R(t)=[Vₓ²(t−t_{b})²+R_{b}²]^{1/2} where R_{b}=y is the closest distance of the scatterer to the transducer path, t_{b}=x/Vₓ is the time at which the transducer is at its closest distance to the scatterer, and Vₓ is the velocity of the transducer, assumed to be constant. Generally the scatterer is only within the angular spread of the transmitted beam if R_{b}>Vₓ|t−t_{b}| and in this case

$$R ( t ) \approx {R}_{b} + \frac{{{V}_{x}^{2} ( t - {t}_{b} )}^{2}}{2 {R}_{b}}$$

is a good approximation. Then the received signal, after down-converting, would be

$${S}_{n}^{r} ( t ) = exp [ πi \frac{{B}_{0}}{{T}_{p}} ( t - {t}_{n} - τ ) - π i \frac{{F}_{c}}{c} ( 2 {R}_{b} + \frac{{{V}_{x}^{2} ( {t}_{n} - {t}_{b} )}^{2}}{{R}_{b}} ) ]$$

where c is the speed of sound in body tissue, which is close to the speed of sound in water, 1540 m/s, and τ is the time, approximately 2R(tₙ)/c, for ultrasound to travel from the transmitter to the scatterer and back to the receiver. The small change in location of the receiver, during the time the ultrasound travels to the scatterer and back, has been neglected. This equation shows only the phase of the received signal, not its amplitude, so the attenuation of the signal, due to spreading out of the transmitted signal, and losses of the transmitted signal in body tissue, have not been included. The time-delayed fast reference function is the complex conjugate of the transmitted modulation function, only including the B₀ term in the phase:

$${S}_{fast}^{ref} ( t ) = exp [ - πi \frac{{B}_{0}}{{T}_{p}} {( t - {t}_{n} )}^{2} ] for \vert t - {t}_{n} \vert < {T}_{p} / 2$$

This form of the fast reference function is optionally used for the case there the transmitted pulse has the form given above, with linear frequency modulation and a square envelope of width Tₚ. If a different envelope or frequency modulation were used for the transmitted pulse, the fast reference function optionally would have the same form as the transmitted pulse. Alternatively, the fast reference function may have a different envelope than the transmitted pulse, chosen for example to reduce undesired side lobes, and/or to reflect changes in shape of a transmitted pulse due to dispersion, or frequency-dependent losses, in body tissue. Sometimes the different envelope is not considered to be part of the fast reference function, but to be the result of a window in the convolution.

Convolving the fast reference function with the down-converted received signal for each pulse n, yields a filtered signal, which may be considered a distribution of echoes, as a function of time delay, for the received signal for that pulse.

pₙ^{r}(τ)exp[iφₙ(τ)]=∫dtS_{fast}^{ref}(t−τ)Sₙ^{r}(t)

To estimate the resolution in time delay that can be obtained with this linearly frequency modulated pulse of bandwidth B₀, consider the received signal for this pulse in the case of a single localized scatterer that is at a distance for which the time delay is τ′. We find

$${p}_{n}^{r} ( τ ) = {T}_{p} sin c [ π {B}_{0} ( τ - {τ}^{′} ) ]$$

$${ϕ}_{n} ( τ ) = - π \frac{{F}_{c}}{c} ( 2 {R}_{b} + \frac{{{V}_{x}^{2} ( {t}_{n} - {t}_{b} )}^{2}}{{R}_{b}} )$$

The amplitude pₙ(τ) is a short, fast oscillating pulse with side lobes of about −13 dB. It can be calculated efficiently using an FFT algorithm, and the side lobes can be reduced by using an appropriate window in the convolution. The sinc function (defined as (1/x) sin(x)) has its first zero when the argument is equal to π, so the resolution in delay time is about 1/B₀, and the resolution in range R is c/2B₀. This distance can be 1 mm or less, for ultrasound pulses with F_{c} about 1.5 MHz, which can penetrate 30 cm or even 40 cm into soft tissue, and bandwidth B₀ more than 50% of F_{c}.

It should be noted that if the transmitted pulse is a short pulse, with B₀Tₚ˜1, instead of a frequency modulated pulse with B₀Tₚ>>1, then the received signal for each pulse may directly provide a measure of the density of scatterers as a function of range, without any need to filter by the fast reference function.

FIG. 3B shows a plot 318 of the envelope 320 of a transmitted pulse, which is a square wave extending over a time interval Tₚ=0.2 msec, and of pₙ^{r}(τ), the filtered received signal 322, for a single scatterer that has a time delay of 0.3 msec. The filtered received signal has a peak that is much more sharply defined than the width Tₚ of the transmitted signal, since the filtered signal has width about 1/B₀, which is about Tₚ/130 for this transmitted pulse. FIG. 3B also shows a plot 324 of the frequency spectrum of the fast reference function S_{fast}^{ref}(t), with the vertical and horizontal axes both having an arbitary scale. Plot 326 of FIG. 3B shows the frequency spectrum of the slow reference function p_{slow}^{ref}(n), also with arbitrary scale (different from plot 324) for the vertical and horizontal axes. The slow reference function, which will be described below, has almost the same form as the fast reference function, though on a very different time scale.

The phase φₙ(τ) is (ignoring the change of position of the transducers during the pulse) independent of τ, but does change slowly, from pulse to pulse, through its dependence on tₙ. This slow change in φₙ can be used to find the image density as a function of x, the position in the direction of the path of motion of the transducers, using spectral analysis to separate components of different Doppler shift that come from different azimuthal directions, and using a slow reference function to convert a signal that is a function of pulse n, to a signal that is a function of lateral position x. To estimate the resolution in x that may be obtained, consider a slow reference function, time delayed by Δt,

$${p}_{slow}^{ref} ( n - Δ t / T ) = exp [ \frac{π i {F}_{c}}{c} ( 2 {R}_{b} + \frac{{{V}_{x}^{2} ( nT - {t}_{b} - Δ t )}^{2}}{{R}_{b}} ) ]$$

where T is the time from one pulse to the next, assumed to be constant, so tₙ=nT. Convolving this slow reference function with exp(iφₙ), the slowly varying part of the received signal, we find

$$p ( Δ t ) = {\sum }_{n} exp ( i {ϕ}_{n} ) {p}_{slow}^{ref} ( n - Δ t / T )$$

It should be noted that the sum over n extends only over the range of n for which the scatterer is within the transmitted ultrasound beam. Assuming that the ultrasound beam has an angular spread of about 90°, and is centered in a direction perpendicular to the direction of motion of the transducer, the range of n in the sum over n is from t_{b}/T−R_{b}/VₓT to t_{b}/T+R_{b}/VₓT. Even if the ultrasound beam had a much broader spread than 90 degrees, it might still be appropriate to include only this range of n in the sum over n, because for n well outside this range, the distance R(tₙ) to the scatterer would be much greater than inside this range, so the echoes for those values of n would be significantly attenuated, and contribute much less to the sum over n. The sum over n can be replaced by an integral over n, to good approximation, if the phase φₙ and the phase of the slow reference function do not change by more than π from one term in the sum to the next, a condition that may be met, for example, if VₓT, the change in location of the transducers between successive pulses, is less than c/2F_{c}, half a wavelength λ of the center frequency F_{c}. We also assume that terms of order λ/R_{b} may be neglected. With these approximations,

$$p ( Δ t ) \approx \frac{2 {R}_{b}}{{V}_{x} T} sin c ( \frac{2 π {F}_{c} {V}_{x} Δ t}{c} )$$

The sinc function goes to zero when VₓΔt is equal to c/2F_{c}, or half a wavelength λ of the center frequency F_{c}, so this is the resolution in x, independent of depth R_{b} beneath the surface, at least when the depth is less than the path length of the transducer. This estimate ignores the finite extent of the transducer in x, and if that is taken into account, the resolution in x is generally about half of the transducer width, again independent of R_{b}. For a 1 mm wide transducer, appropriate to use for 1.5 MHz ultrasound, the lateral resolution can be as fine as 0.5 mm Having a lateral resolution independent of depth is different from prior art ultrasound imaging methods, where the lateral resolution gets worse at increasing depth R_{b}, in proportion to depth. Having an effective larger aperture at greater depth R_{b}, with contributions to the signal from pulses transmitted from and received at a broader range of positions xₙ, allows the resolution in x to be independent of depth, in spite of the fact that a given resolution in x corresponds to a finer angular resolution at greater depth. The longer exposure to the beam, of a given location in the imaging region, at greater depth, also partly compensates for the greater attenuation of echoes coming from greater depth.

To describe how the received signal is used to calculate the image, it will be convenient to express the fast filtered amplitude pₙ^{r}(τ) and phase φₙ(τ), as functions of range R=cτ/2, instead of τ, and for convenience we use the same symbols for those functions, pₙ^{r}(R) and φₙ(R),

The fast filtering is optionally done for every pulse n, and for each of a set of values of R covering a range of distances corresponding to the time range when the receiver is active after each pulse, and spaced to provide a desired resolution in the image. Optionally, in order to compensate for attenuation of the received ultrasound, pₙ^{r}(R) is now divided by an attenuation factor A(R), which takes into account attenuation due to spreading out of the ultrasound, as well as due to losses in body tissue. Alternatively, the fast reference function is defined to include this factor.

At 306, pₙ^{r}(R)exp[iφₙ(R)] for each value of R is spectrally analyzed in tₙ, the time at which the nth pulse is transmitted and received, for example performing a Fourier analysis

$$\tilde{p} ( ω , R ) = {\sum }_{n} exp [ i ω {t}_{n} + i {ϕ}_{n} ( R ) ] {p}_{n}^{r} ( R )$$

for a set of values of Doppler frequency shift ω. Each value of Doppler shift ω corresponds to a component of the signal coming from a different azimuthal angle with respect to the direction of motion of the transducer. The values of to range, for example, from −π/T to +π/T, where T is the spacing between adjacent tₙ's, and the values of ω are spaced, for example, every 2π/NT, where NT is the total range of xₙ's. The calculation is done, for example, using an FFT algorithm. Alternatively, instead of using a Fourier transform, other transforms are used for the spectral analysis, for example various wavelet transforms. In all these cases, the steps described below may have to be appropriately modified, in order to end up with the image density as a function of x and y.

The transformed signal \tilde{p}(ω, R) represents total signal received from scatterers at a distance R from the transducer, with a Doppler shift given by ω. The speed Vₓ of the transducers is assumed to be constant. The signal for a given to comes largely from scatterers that are close to an angle arcsin(ωλ/4πVₓ) from the transmitter and receiver, relative to the y direction, normal to the surface of the body, where λ=c/F_{c} is a wavelength at the center frequency F_{c}. This fact can be used to express R in terms of the depth R_{b}, and ω, for example

$$R = {{R}_{b} [ 1 - {( \frac{ω λ}{4 π {V}_{x}} )}^{2} ]}^{- 1 / 2}$$

At 308, a range migration algorithm is optionally applied to \tilde{p}(ω, R), transforming it from a function of range R and Doppler shift ω to a function \tilde{q}(ω, R_{b}) of depth R_{b} and Doppler shift ω, for example using the relation between R, ω, and R_{b} given above. Alternatively, an approximation is used, for example

$$R = {R}_{b} [ 1 + \frac{1}{2} {( \frac{ω λ}{4 π {V}_{x}} )}^{2} ]$$

Alternatively, instead of using a range migration algorithm, a backprojection algorithm is used, particularly if the transducer is not moving at a constant speed Vₓ. Backprojection algorithms are described, for example, for use in synthetic aperture radar (SAR), in Buxa et al, “Mapping of a 2D SAR Backprojection Algorithm to an SRC Reconfigurable Computing MAP Processor,” downloaded from www.srccomp.com/techpubs/docs/HPEC05_SARBackprojection.pdf, on May 21, 2012, and in the references cited therein, G. W. Carrara, R. S. Goodman, R. M. Majewski, Spotlight Synthetic Aperture Radar, p. 495-499, Norwood, Mass.: Artech House, Inc., 1995, and L. Gorham, K. D. Naidu, U. Majumder, and M. A. Minardi, “Backhoe 3D ‘Gold Standard’ Image,” Algorithms for Synthetic Aperture Radar Imagery XII, Vol. 5808, SPIE DSS, Orlando, Fla., Mar. 28-31, 2005. Backprojection algorithms have the potential advantage that they may produce more accurate images, and they do not require any assumption about Vₓ being constant. Range migration algorithms have the potential advantage that they are generally much less computationally intensive, and easier to implement.

At 310 the signal \tilde{q}(ω, R_{b}), at each value of R_{b} in a set of values (for example, the same set of values used for R), is filtered by a slow reference function \tilde{S}_{slow}(ω, R_{b}), for example

$${\tilde{S}}_{slow} ( ω , {R}_{b} ) = exp [ \frac{4 π i {R}_{b}}{λ} - \frac{i {R}_{b} λ {ω}^{2}}{8 π {V}_{x}^{2}} ]$$

in order to convert the signal \tilde{q}(ω, R_{b}) from a function of frequency in tₙ to a function of frequency in t_{b}. This expression for the slow reference function is based on the approximation that the ω^{t} term in the exponential is small, assumes a constant speed Vₓ of the transducer, and neglects the finite angular spread of the transmitted ultrasound, and the greater attenuation of ultrasound reflecting from greater ranges. These approximations give the slow reference function a particularly simple form, but other forms for the slow reference function are alternatively used, and the form of \tilde{S}_{slow}(ω, R_{b}) is optionally modified if the speed of the transducer is known not to be constant, for example due to known acceleration and deceleration periods in the case of a linearly moving transducer, or circular motion in the case of a circularly moving transducer. Optionally, after taking ultrasound data for a slice, and before reconstructing an image, \tilde{S}_{slow}(ω, R_{b}) is corrected for differences in the expected position of the transducer as a function of pulse number, that were detected by a position or velocity sensor while the ultrasound data were taken.

At 312, the resulting signal \tilde{D}(ω, R_{b})=\tilde{S}_{slow}(ω, R_{b}) \tilde{q}(ω, R_{b}) is inverse Fourier transformed at each value of R_{b}, and at each of a set of values of t_{b} (chosen, for example, based on the range of t_{b} in the imaging region, and on a desired resolution of the image in the x-direction), to produce an image density D(t_{b}, R_{b}) which is a function of t_{b} and R_{b}, for example,

D(t_{b},R_{b})=∫dωexp(iωt_{b})\tilde{D}(ω,R_{b})

It will recalled that R_{b}=y and t_{b}=x/Vₓ, so D(t_{b}, R_{b}) provides an image density as a function of a pixel location (x, y). It should be noted that normalization factors have generally not been included in these expressions for the received signal in its raw and processed forms, since in any case the image density may be expressed in arbitrary units. It should be understood that “pixel” is used herein as a generic term that includes voxels. The pixel densities found for a two-dimensional slice image, for example, may become voxel densities if several such slices are combined to form a three-dimensional image.

Optionally, the signal processing is done storing the signal, at each stage of the processing, in a matrix form, i.e. in a 2-D array of computer memory, with the rows and columns of the matrix representing different variables at different stages of the calculation. For example, they represent n and t initially, then n and R, then ω and R, then ω and R_{b}, then t_{b} and R_{b}. The choice of how many bins to choose for each of these variables, which will determine the number of pixels (t_{b}, R_{b}) in the image, is made, for example, based on the maximum resolution possible given F_{c}, B₀, V, and T, as well as on a desired signal to noise ratio in the image.

At 314, the absolute value of the complex image density D(t_{b}, R_{b}) is optionally found for each pixel (t_{b}, R_{b}) in the image, and at 316 the image is optionally displayed, or stored for later use. In some embodiments of the invention, the complex image density D(t_{b}, R_{b}) is saved, and used, for example, to reduce noise by coherently adding up images of the same imaging region taken repeatedly. In some embodiments of the invention, where multiple slices of a three-dimensional imaging region are imaged, the pixel densities for the next slice are now calculated, if this is not the last slice. Optionally, the completed slice image or 3-D image is transferred to a post-processor using, for example, the DICOM standard of medical communication.

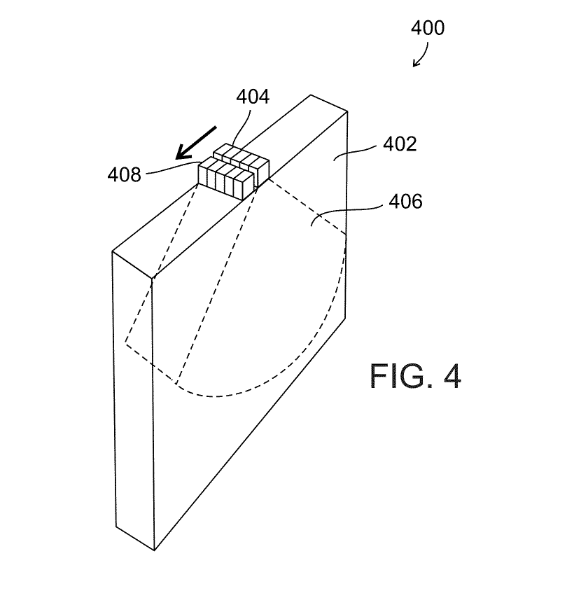

FIG. 4 shows a system 400, similar to system 100, for reconstructing ultrasound images of an imaging slice 402, of body tissue. In order to keep most of the transmitted ultrasound waves within the finite width of the slice, transmitting transducer 404 comprises an array of transducer elements, arranged along the thickness of the slice. By adjusting the relative phase of the different elements in the array, an ultrasound beam 406 can be kept from spreading out too much in a direction normal to the slice. Optionally, the ultrasound is even focused slightly inward initially, to minimize how far it spreads outside the slice. Within the plane of the slice, however, ultrasound beam 406 has a large angular spread, similar to system 100.

Receiving transducer 408 also comprises an array of transducer elements, arranged across the thickness of the slice. By adding up the signals from the different elements with appropriate phase differences, the receiver can remain sensitive to ultrasound reaching it from within the slice, over a broad range of angles in the plane of the slice, but insensitive to ultrasound coming from outside the slice. The combination of a transmitter that transmits ultrasound mostly within the slice, and a receiver that receivers ultrasound mostly from within the slice, means that the reconstructed image may have very little interference from scatterers outside the slice.

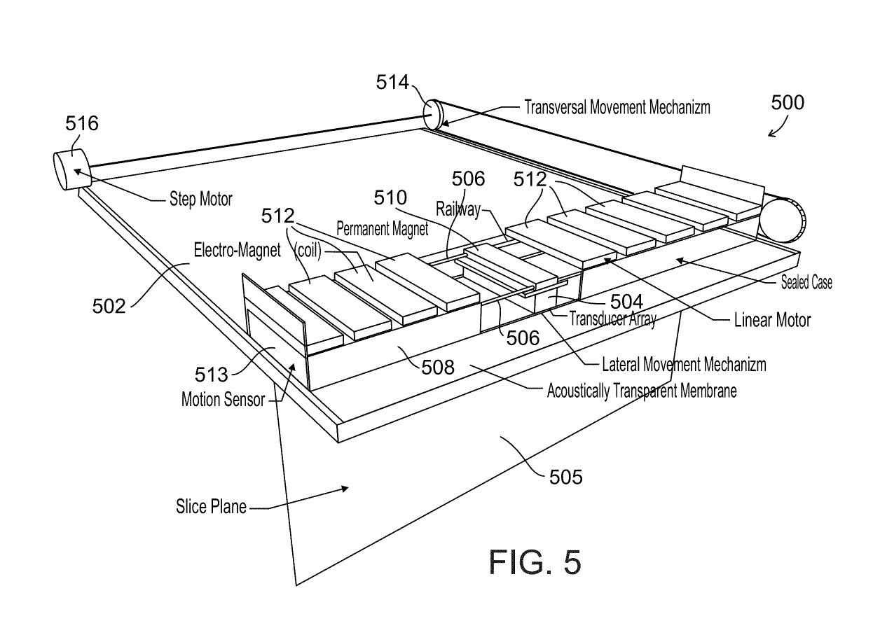

FIG. 5 shows an ultrasound imaging system 500 that produces two-dimensional images of a series of parallel slices of a three-dimensional imaging region, which can be combined at the end to produce a three-dimensional image. System 500 is mounted on an acoustically transparent plate 502 that is in good acoustic contact with an extended area of the body being imaged. A transducer array 504 includes a transmitting part that transmits ultrasound into an imaging slice 505, and a receiving part, the same as or different from the transmitting part, that receives echoes of the transmitted ultrasound from the imaging slice, and uses a received signal of those echoes to reconstruct an image of slice 505. Transducer array 504 is mounted on rails 506 which run in a direction of motion of transducer array 504, and the array and rails are enclosed in a sealed case 508. Optionally, sealed case 508 is filled with a liquid, optionally degassed, that provides acoustic contact between the transducer array and plate 502. Degassing the liquid may be useful to reduce heating of the liquid by the ultrasound, and consequent formation of large bubbles, especially in polar liquids such as water, which tend to dissolve gases from their environment. It may be less important in nonpolar liquids, such as hydrocarbons, which have less of a tendency to dissolve gases. Transducer array 504 is moved back and forth along the rails by a motor comprising a permanent magnet 510 mounted on top of the transducer array, and a series of coils 512 arranged along the track. Optionally case 508 is made of a diamagnetic or at least non-ferromagnetic material. By putting current in a proper direction through the coils in front of and/or behind the transducer array, the coils in front can pull the transducer array, and/or the coils behind can push the array, thereby moving it along the rails. Having the coils all in the stationery part of the motor, and permanent magnet in the moving part, has the potential advantages that it avoids the need to bring current to a moving part, and reduces possible electromagnetic interference with the operation of the transducer, both for transmitting and for receiving.

In an exemplary embodiment of the invention, a sensor 513 senses the position and/or velocity of the transducer array along the rails, as a function of time. For example, an optolectronic sensor is optionally used, mounted on the transducer array, and senses the position by reading ticks on the inside wall of the case. Alternatively, a laser range sensor or Doppler sensor is used, for example mounted on the end of the case and looking toward a target on the transducer array. Optionally, the sensor can detect errors in position of the transducer array corresponding to errors of 5 degrees, 2 degrees, 1 degree, or 0.5 degrees in phase φₙ. Optionally, the sensor can detect errors in position of 25 μm, or 10 μm, or 5 μm, or 2 μm.

Once ultrasound receiver data has been recorded for slice 505, the whole assembly comprising the motor, sealed case, and transducer array is optionally moved in a direction transverse to its length, by transverse movement mechanism 514, to take receiver data for another slice, parallel to slice 505. Mechanism 514 comprises a pulley that pulls the motor, case, and transducers along, and is driven by a stepper motor 516. Alternatively, the assembly is moved manually, to image a new slice. Once the transducer array has taken receiver data for all of the slices into which the three-dimensional imaging region is divided up, and a 2-dimensional image is reconstructed for each slice, the images of the different slices are optionally assembled into a three-dimensional image of the entire imaging volume.

System 500 optionally has a linear motor that can move the transducer over a path 1 cm long, or 2 cm long, or 5 cm long, or 10 cm long, or 20 cm long, or a smaller, intermediate or larger distance. The width of system 500 in the transverse direction, which the linear motor and transducer move in in order to image different slices, is also optionally 1 cm, or 2 cm, or 5 cm, or 10 cm, or 20 cm long, or smaller, intermediate or larger distances. A size of 20 cm by 20 cm, for example, may be useful for imaging all or much of the abdomen. Smaller sizes are useful for imaging smaller regions of the body. Optionally, the length of the path that the transducer moves over, and/or the width of system 500 in a transverse direction, is 10 times a wavelength of ultrasound, in the body, for the average frequency of ultrasound used, or 20, 50, 100 or 200 times the wavelength, or a smaller, intermediate, or greater number.

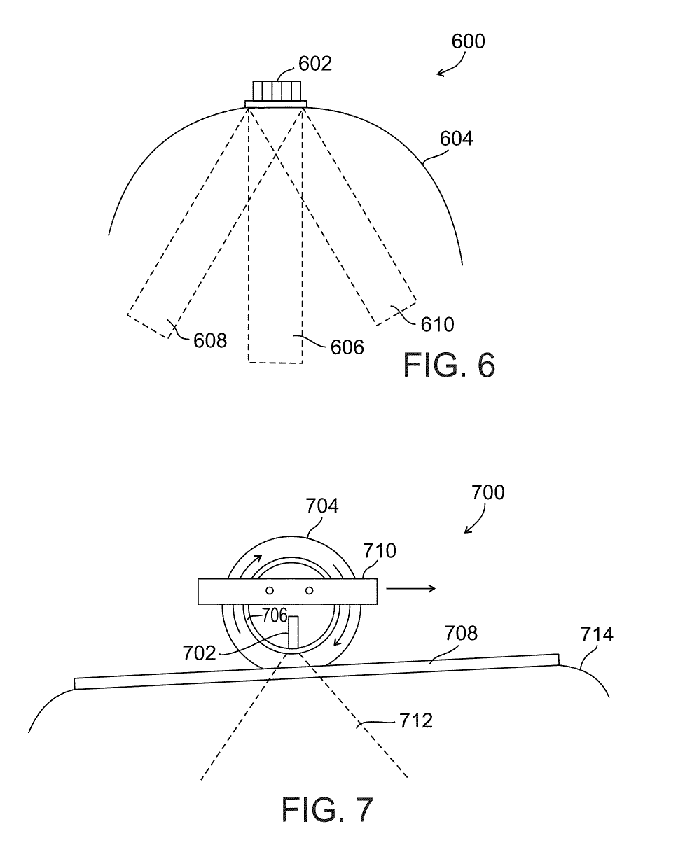

FIG. 6 shows an alternative system 600 for producing a three-dimensional image from slices, but using electronic steering, instead of physical motion of the transducers, to change to a different slice. A transducer array 602, similar to array 504 in FIG. 5, transmits ultrasound waves into an imaging slice 606, and moves perpendicular to the plane of the drawing, along the plane of slice 606, which is also perpendicular to the plane of the drawing, producing an image of slice 606 as described in FIGS. 1A-5. The phases of the elements in the transducer array are, for example, all the same, or vary slightly with position in order to produce a slightly focused beam, that will be confined largely to slice 606. Similarly, the signals produced by the different elements of the receiver array, which optionally is the same array when it is switched to acting as a receiver, are added up in phase, so that the array will be most sensitive to reflected ultrasound coming from slice 606. In order to image a different slice, the elements of the transducer array have a phase that varies approximately linearly across the transducer, so as to steer the ultrasound waves in a different direction, into imaging slice 608, for example. Similarly, the receiving array adds the signals of the different elements together with a phase that varies linearly across the transducer, so that the transducer is sensitive mostly to reflected ultrasound coming from slice 608. The transducer array moves perpendicular to the plane of the drawing, optionally back in the opposite direction to its motion while imagining slice 606, while it is imaging slice 608. A third slice 610 can then be imaged by having the phases of the different elements change with position in the opposite direction, and the procedure is optionally further repeated for any number of slices desired.

FIG. 7 shows an ultrasound imaging system 700, similar to system 500 in FIG. 5, but with a cylinder 704 that acoustically couples a transducer array 702, seen edge on, and plate 708, and that rolls along the plate without slipping, potentially reducing drag and allowing a more uniform motion. A coupling element 710, rigidly attached to transducer array 702, pulls transducer array 702 to the right, while keeping it pressed against a low friction inner surface 706 of cylinder 704. Inner surface 706 is, for example, a thin hydrophilic layer. This causes cylinder 704 to roll along plate 708 without slipping. Cylinder 704 is made of a compressible material, such as rubber, that will not slide easily along surface 708, especially when it is pressed against surface 708 by coupling element 710. This pressure causes the surface of cylinder 704 to deform slightly, where it touches plate 708, producing a finite area of contact that allows good acoustic coupling between transducer array 702 and plate 708. Transducer array 702 transmits an ultrasound beam 712 into body 714, and receives echoes from body 714, through cylinder 704 and plate 708. Optionally, the contact area is at least wide enough to accommodate the full desired angular spread of the transmitted ultrasound beam, and the full desired angular range of sensitivity of the receiver.

System 700 may be especially useful for non-destructive testing of large objects, such as aircraft, because it can be easily adapted to roll cylinder 704 over a large distance.

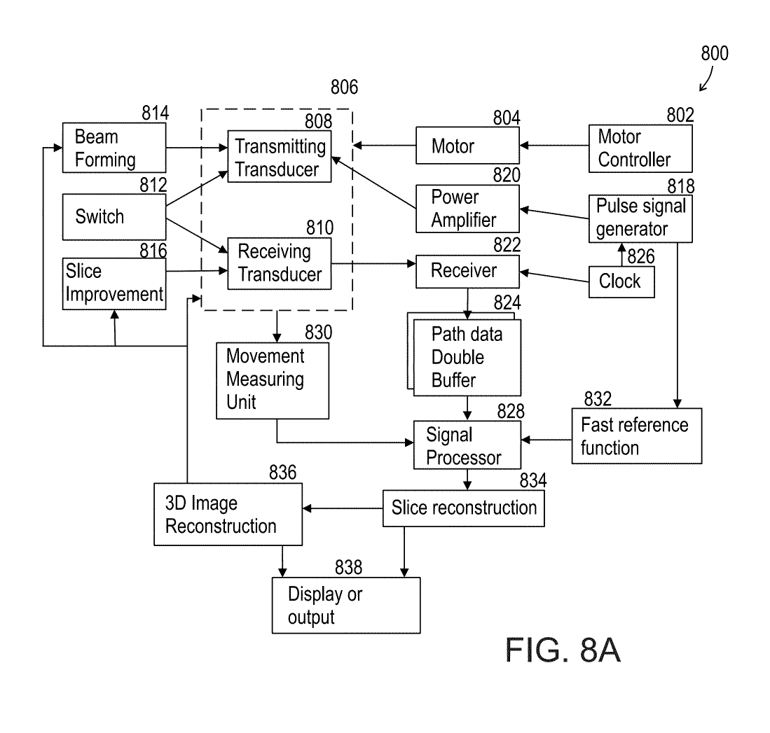

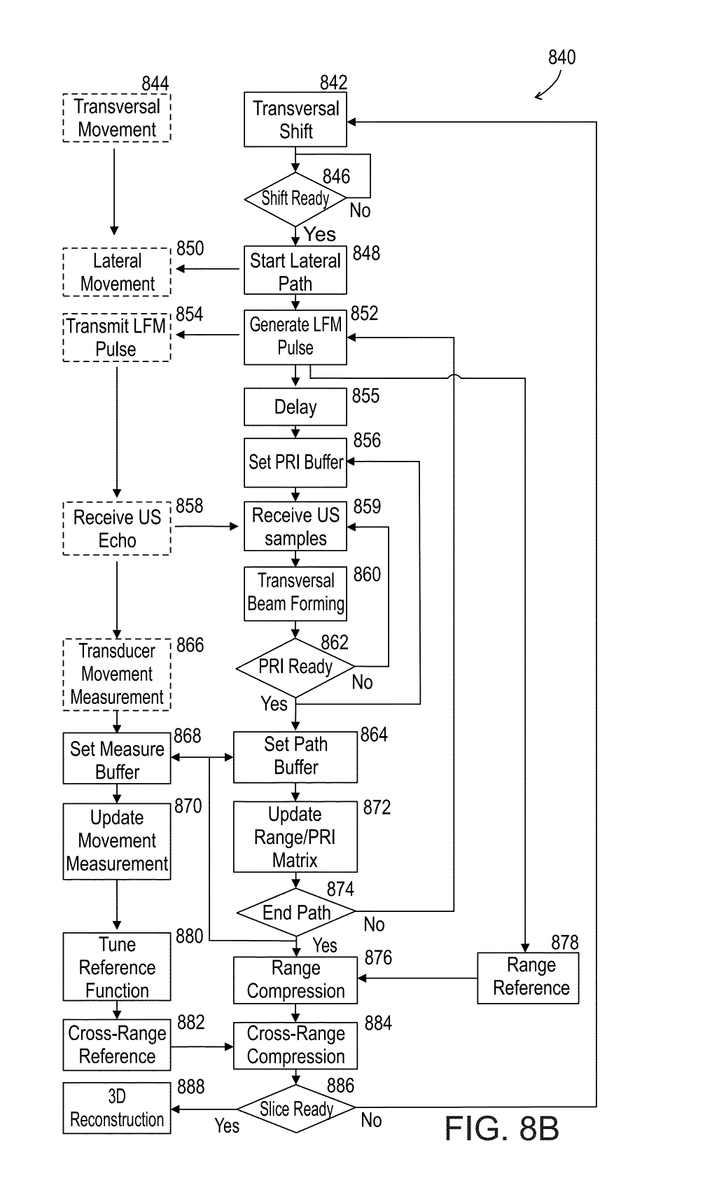

FIG. 8A shows a block diagram 800 of the architecture of a controller, such as controller 120 in FIG. 1A, used in any of the foregoing ultrasound imaging systems. A motor controller 802 controls a motor 804, that moves a transducer array 806, including a transmitting transducer 808 with one or more elements, and a receiving transducer 810 with one or more elements, that may be the same as or different from the transmitting transducer. A switch 812 switches the transducer from being connected to transmitting circuitry and receiving circuitry, if the same transducer is used for transmitting and receiving. Even if different transducers are used, switch 812 optionally disconnects the receiving transducer while the transmitting transducer is transmitting, in order to avoid damage to the receiving circuitry. A beam forming module 814 optionally adjusts the phases of different transmitting transducer elements, in order to form a beam that is relatively well confined to the thickness of an imaging slice. A slice improvement module 816 optionally adds up signals from the receiver transducers elements with different relative phases, so as to limit the sensitivity of the receiving transducer to ultrasound at least fairly well to echoes coming from the imaging slice.

The transmitting circuitry comprises a pulse signal generator 818, which generates a desired pulse shape, such as a fast linearly frequency modulated pulse. This signal is fed to a power amplifier 820, for example a linear power amplifier, which drives the transmitting transducer, through impedance matching circuitry. Optionally, the power amplifier is replaced by a much less expensive three-level excitation amplifier, which can only produce three output voltages, +V, −V, and 0. Such an amplifier can still drive an ultrasound transducer to produce a linearly frequency modulated pulse which is close to that produced by a linear amplifier, because of the limited bandwidth of the transducer response to the signal. Such a three-level amplifier generally has much higher power efficiency than a linear amplifier, and using such a three-level amplifier greatly decreases the memory requirements for the signal generator.

The receiver circuitry comprises receiver module 822, which, for example, converts the receiver signal into digital form and down-converts it. so it can be stored in path data double buffer 824. A stable clock, such as local oscillator 826, controls the timing of the signal generator and the receiver, making it possible to obtain accurate data on the phase of the received signal relative to the transmitted signal. The data in double buffer 824 is manipulated by signal processor 828, for example as described above in FIG. 3. A movement measuring unit 830 provides input to signal processor 828 about the exact position of the transmitting and receiving transducers as a function of time, allowing adjustments to be made in the signal processing, for example in the phase of \tilde{S}_{slow}(ω, R_{b}), due to irregularities in the motion. A fast reference function 832, based on the pulse shape generated by pulse signal generator 818, is also used in the signal processing, as described in FIG. 3.

Finally, slice reconstruction module 834 displays and/or stores an image of the slice resulting from the signal processing, and a 3D image reconstruction module 836 optionally combines multiple slice images into a 3D image. The 3D image reconstruction module also optionally controls the motion or electronic steering of the transmitting and receiving transducers, in order to image the next slice after each slice is completed. A display or output module 838 displays or otherwise outputs the slice images and/or the 3-D image.

FIG. 8B shows a flowchart 840, for the control software used for obtaining a 3-D image from system 500. At 842, the software initiates a shift of sealed case 508, together with the transducer array and linear motor, in a transverse direction to a new slice, if it is not already situated over the first slice to be imaged. This is done by activating transversal movement module 844, which controls stepper motor 516. At 846, a check is made if the shift has been completed, and if so, the motion of the transducer along its lateral path is initiated at 848, using a lateral movement module 850, that operates the linear motor. At 852, a pulse is generated, for example a fast linearly modulated pulse, and the pulse is transmitted by the transmitting circuitry and transducer at 854. A delay in the pulse transmission, long enough to receive echoes of the transmitted pulse, is initiated at 855. The pulse repetition interval (PRI) buffer, which holds the received signal for one pulse, is set at 856, and receiver data, obtained from the receiving circuitry and transducer at 858, is stored in the PRI buffer at 859. At 860, the data from the different elements in the transducer array is combined by a transversal beam forming module, to eliminate echoes that do not come from a direction within the slice, and the resulting data is stored in the PRI buffer. A check is made at 862 to see whether all receiver data for that pulse has been received. When it has been, the path buffer, which stores all the receiver data, already processed by the beam forming module, for that pulse, is set at 864. Meanwhile, at 866, data on the position and/or velocity of the transducer is received from sensor 513, and a measurement buffer is set at 868. The measurement data for the position of the transducer at this pulse is updated at 870, and together with the received signal data in the path buffer, is added at 872 to a range/PRI matrix which holds the processed receiver data as a function of time for each pulse. At 874, a check is made to see whether the transducer is at the end of its path for this slice. If not, the next pulse is generated by the pulse generator at 852. When the last pulse has been generated and its received data stored in the matrix, a range compression module at 876 filters the received data for each pulse by filtering it with a fast reference function, using a range reference function stored at 878, which is based on the transmitted pulse generated by the pulse generator. After filtering with the fast reference function, the range/PRI matrix has data stored as a function of pulse number and range, rather than pulse number and time. At 880, the slow reference function is tuned, based on the measurement data about the position of the transducer during each pulse. At 882, a slow reference function is generated, also called a “cross-range reference function” because it is used to separate data from different lateral positions, and is applied to the data in the range/PRI matrix, by a cross-range compression module at 884. This module performs the actions described at 306, 308, 310 and 312 in flowchart 300, in FIG. 3. After performing this processing, the data in the range/PRI matrix is a function of depth y and lateral position x, that is to say it is a matrix of the image densities of the pixels in the slice. At 886, a check is made to see if all the slices have been processed. If not, the next slice is processed, starting at 842, initiating a movement of the transducer and linear motor to the next slice to be imaged. When the last slice has been imaged, the imaging data for all the slices are combined to form a 3-D image at 888.

### Circular Moving System

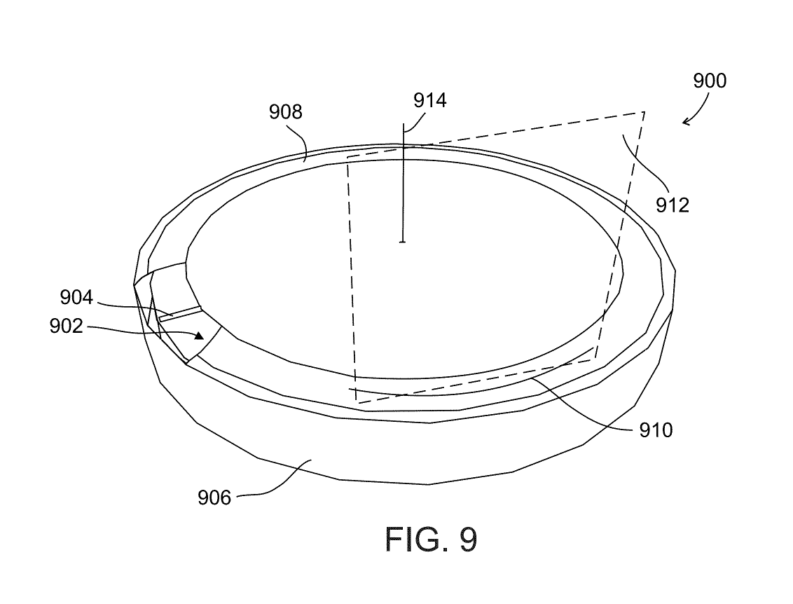

FIG. 9 shows a circular moving transducer assembly for producing ultrasound images, similar to system 500 in FIG. 5, but with a circularly moving transducer 904 instead of a linearly moving transducer. A rotating part 902 in the shape of a circular disk has a transducer or transducer array mounted in it. A potential advantage of using only a single transducer rather than an array is that it will have a thicker slice, which may be needed to accommodate the circular path of the transducer. The rotating part is surrounded by a static part 906, including an acoustically transparent membrane 908 that plays the same role as the plate in the linearly moving system, acoustically coupling the transducer to the body. A liquid, optionally degassed, optionally surrounds the rotating part, acoustically coupling the moving transducer to membrane 908, while keeping the drag relatively low and uniform. A circular path 910 of the transducer extends for only a fraction of the circumference of the assembly, for example no more than 60 degrees, and within this angle the transducer path is close enough to a straight line that it can be used to image a flat slice 912 that is thick enough to accommodate the curvature of the path. Optionally, only a part of membrane 908 is in contact with the body, including a part that includes the circular arc path where imaging is done. This may be done, for example, in order to use a large imaging slice, for example 20 cm across, while extending no more than about 60 degrees around the circumference of the assembly. That would require the entire assembly to be at least 40 cm in diameter, which might be too wide to fit entirely on a surface of the body, in the region where imaging is being done.

The image reconstruction is done in a way similar to the linear motion system. The rotating part rotates around a rotation axis 914, and is driven by a motor.

System 900 need not be limited to obtaining imaging data for only one slice, covering up to 60 degrees, for each rotation of the rotating part. Optionally, additional slices are imaged in a given rotation of the rotating part. Each slice is, for example, a planar slice, with some thickness, which includes an arc of the circular path of the transducer, over an angle up to about 60 degrees, extending straight into the body. Six or more such slices are optionally imaged in a single rotation, even without overlap of the slices. Optionally, the slices overlap to some extent, and data from one or more pulses is used for reconstructing two different imaging slices. In some embodiments of the invention, multiple transducers, at different radial positions, are used to image slices at different radial positions.

The circular motion system has the potential advantage that it may be more robust and accurate than the linear motion system. The rotating part is preferably constructed to be very rigid and stable, for example made out of a hard plastic, and sealed into a cavity of the static part, so the position of the path of the transducer is well fixed once the assembly is pressed against the body. It may be easier to move a rotary motor at a constant speed than a linear motor, since the linear motor has to slow down and stop at the end of each path, and this may make the image reconstruction easier and more accurate. It also may make it unnecessary to have a position or velocity sensor for the transducer array, in the circular case, and it may make the design of the circular motion system simpler, which may make it easier to design a portable system in the circular motion case. Furthermore, it may be much less expensive to scale up a circular motion system than a linear motion system, because a large part of the cost of a linear motion system may be in the linear motor, which has a cost that increases linearly with the length of the path, while the rotary motor used in the circular motion system may be a relatively small part of the cost of the system, and in any case can be scaled up in diameter without making it much more expensive. On the other hand, if the slice only extends for a distance about equal to the radius of the circular motion system, the diameter of the circular motion system may be about twice the length of the linear motion system, for the same slice size.

System 900 is optionally 2 cm, 5 cm, 10 cm, 20 cm, or 30 cm in diameter, or a smaller, intermediate or larger size, depending on how wide a region is being imaged. The larger sizes may be useful for imaging all or much of the abdomen. Optionally the diameter is 20, 50, 100, 200 or 300 times the wavelength, in the body, or ultrasound of the average frequency used, or a smaller, intermediate or greater number.

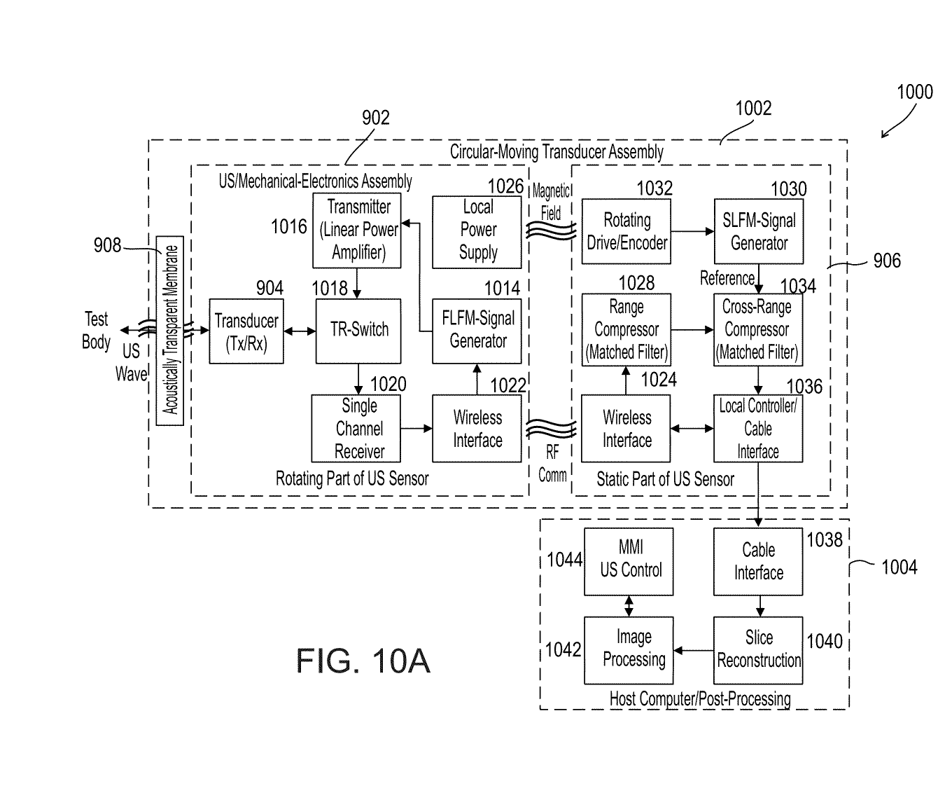

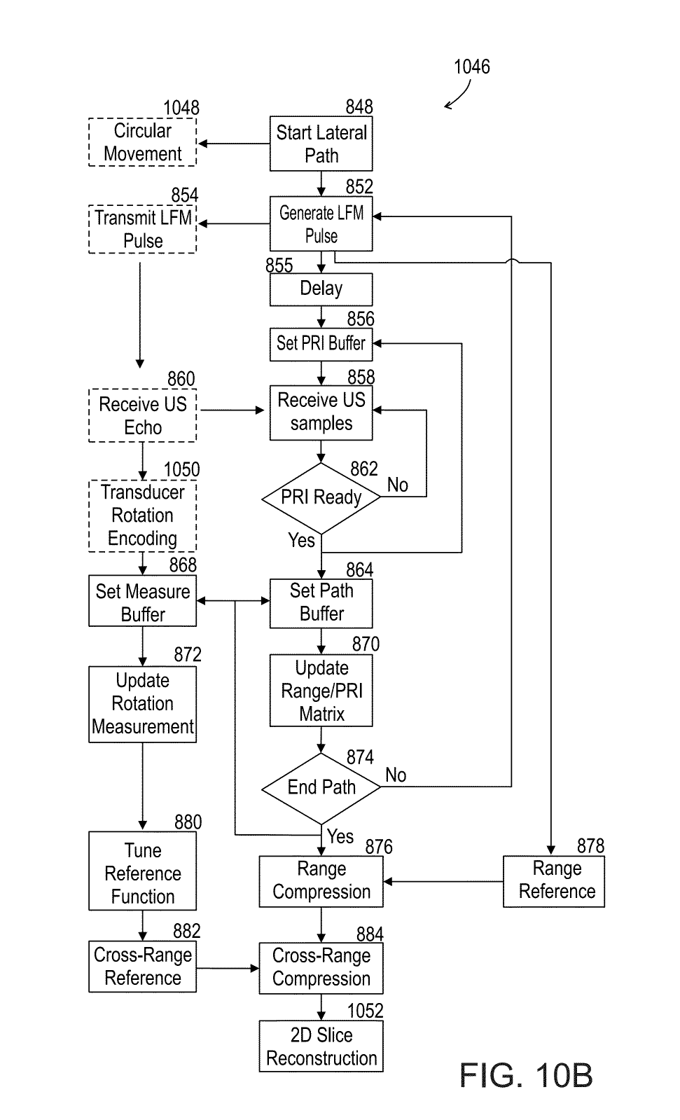

FIG. 10A is a block diagram 1000, showing the different parts of the circular moving transducer assembly 1002, indicating which modules of the system are located in rotating part 902, which are located in static part 906, and which are located in a separate external host computer and post-processing unit 1004. In general, transducer 904, and the supporting circuitry that is small and light, and is best to keep close to the transducer, are located in the rotating part, while larger modules, that need not be in immediate contact with the transducer, are located in the static part, and still larger modules are located remotely. However, in some embodiments of the invention, some of the modules that are located in the static part in FIG. 10 are located in the rotating part, and vice versa, while some of the modules that are located in the host computer in FIG. 10 may be located in the static part or even in the rotating part, and vice versa. The device need not use a separate host computer at all. The static and rotating parts also have means for communication between them, and optionally for power transfer as well.

The transmission and receiving circuitry, that is located in the rotating part, include a fast linear frequency modulated signal generator 1014 and a linear power amplifier 1016 for the transmitter, a transmit/receive switch 1018, and a single channel receiver 1020. Wireless interface 1022 communicates with wireless interface 1024 in the static part. The wireless interfaces need only be able to communicate over a distance of a few centimeters, for example over 2 centimeters. A local power supply 1026, in the rotating part, optionally comprises a rechargeable battery, capacitor, or other energy storage device that is charged up inductively when the motor rotates the rotating part. Alternatively, local power supply 1026 runs off a battery or other storage device that is not charged up while the rotor is rotating and the system is operating, but is charged up and/or replaced periodically, for example by a power cable, when the rotor is not rotating. Alternatively, the rotating part has no energy storage device, and its power consuming parts run directly off the generator, only when the rotating part is rotating and the generator can produce power.

Other modules in the static part are a fast matched filter (“range compressor”) 1028, a slow linear frequency modulated signal generator 1030, and a slow matched filter (“cross range compressor”) 1034, all for signal processing. A rotating drive and encoder 1032 controls the motor that rotates the rotating part, and also measures the rotation and sends information to SLFM signal generator 1030, to adjust the slow matched filters in response to differences in the rotation rate. There is also a local controller/cable interface 1036 to send data back and forth to the host computer. Optionally, another means of communication is used, for example wireless. If a cable is used, and the host computer is not very small and portable, then it is potentially advantageous to use a cable that is long and flexible enough so that the circular moving transducer assembly can be put on a patient's body,

The external host computer includes a cable interface to the static part, a slice reconstruction module 1040, and image processing module 1042 for post processing of the image, and an ultrasound control 1044, to provide a user interface.

FIG. 10B is a flowchart 1046, for the control software used for obtaining a 2-D slice image from system 900. Flowchart 1046 is similar to flowchart 840, the flowchart for the control software for the linearly moving system, and only the differences will be described here. In system 900, the transducer is fixed at a certain radius, so there is nothing like stepper motor 516 in system 500, which moves the transducer to different transverse positions to image different parallel slice. So flowchart 1046 only describes imaging a single slice, although, as will be discussed below, it is possible to image more than one slice with system 900. Initiating motion along the lateral path of the transducer, at 848, is done by controlling a circular movement motor at 1048, rather than a linear motor as in flowchart 840. Instead of measuring the linear position or velocity of the transducer, as is done at 866 in flowchart 840, the angular position or velocity of the transducer, in its circular motion, is measured at 1050. And instead of storing the 2-D imaging data from each slice and combining it into a 3-D image as in flowchart 840, only the image from one slice is reconstructed at 1052.

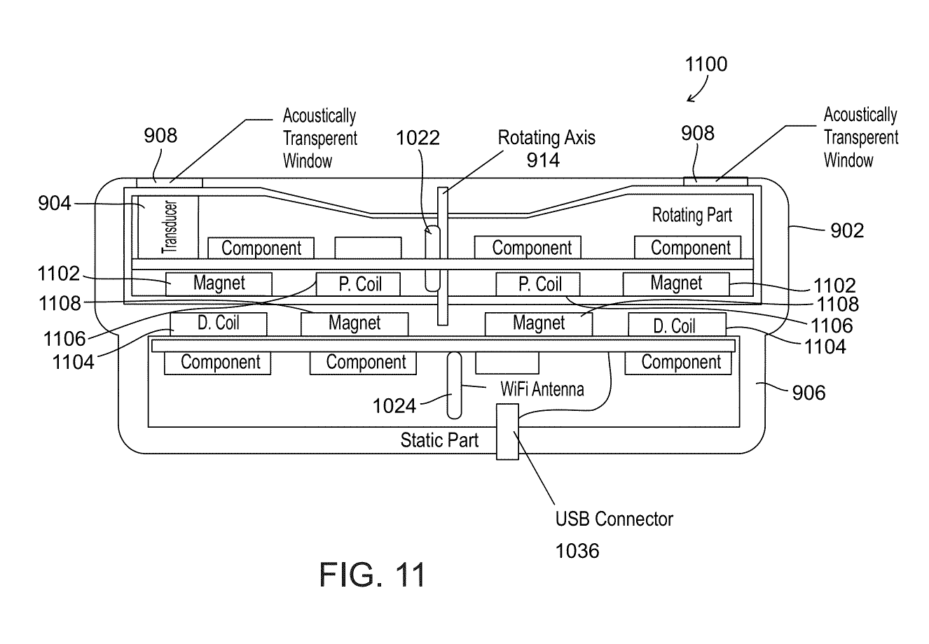

FIG. 11 shows a side cross-sectional view 1100 of the interior of the circular moving transducer assembly. Transducer 904 is near acoustically transparent membrane 908, which goes around the top of the rotating part, although it is static itself. The motor driving the rotation comprises permanent magnets 1102 on the rotating part and driving coils 1104 on the static part. AC current in the driving coils has a frequency and phase such that the force between the driving coils and the adjacent magnets tends to exert a torque on the rotating part, enough to overcome drag and keep it rotating at a constant rotation speed that allows image reconstruction. There are also power coils 1106 in the rotating part, and permanent magnets 1108 across from them in the static part, which comprise a generator that charges up local power supply 1026 in the rotating part, when the motor is operating. The generator imposes an additional drag on the motor, beyond the mechanical drag, including the drag on the transducer by the surrounding liquid. Optionally, as in FIG. 11, the generator coils and magnets are located closer to the axis of rotation than the motor coils and magnets, so that if similar coils and magnets are used for the motor and the generator, the maximum drag torque exerted by the generator will be substantially less than the drive torque exerted by the motor. An axle 1022 holds the rotating part in place, and houses a WiFi antenna for wireless communication between the rotating part and the static part. The static part has its own WiFi antenna 1024, for wireless communication. A cable interface 1036, for example a USB connector, provides a connection to host computer 1004. The blocks labeled “Component” in the rotating part and the static part in FIG. 11 represent any of the electronic components respectively shown in the rotating part and the static part in FIG. 10.

### Pseudo-Motion System

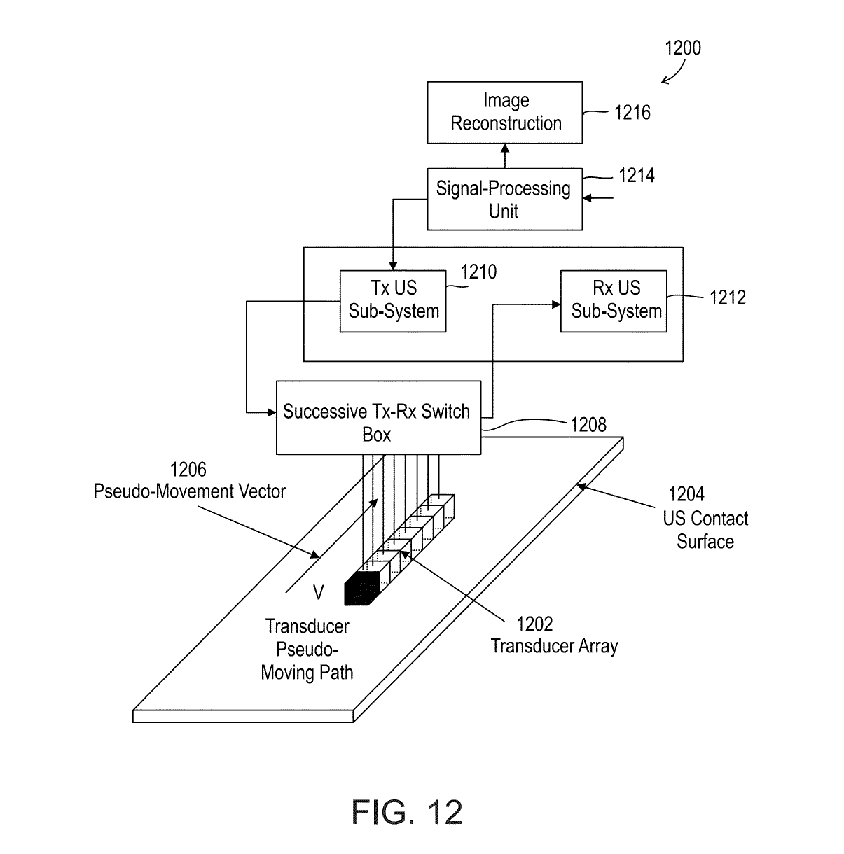

FIG. 12 shows another type of system for ultrasound imaging, operating on the same general principles as the linear moving system and circular moving system described above, but with simulated instead of real motion of the transducers. This pseudo-motion system 1200 comprises a long array of transducers 1202, optionally resting on an acoustically transparent surface 1204, playing the same role as the path followed by the moving transducers in the linear and circular moving systems. The linear and circular moving systems reconstruct an image by measuring ultrasound reflections from the same imaging region, with ultrasound transmitted and received over an extended range of different locations. The different time delays of the ultrasound, coming from different locations, provides the information for reconstructing the image. In pseudo-motion system 1200, instead of a moving transducer there is array of transducers 1202, which are turned on and off sequentially one at a time by successive transmit/receive switch box 1208, simulating the motion of a single transducer, as shown by a “pseudo-movement vector” 1206. The switch box initially connects a first transducer in the array to transmitting circuitry 1210, so it acts as a transmitter for the first pulse, and connects a second transducer in the array to receiving circuitry 1212, so it acts as a receiver for the first pulse. Switch box 1208 then disconnects the first transducer, connects the second transducer to the transmitting circuitry, so it now acts as a transmitter for the second pulse, and connects a third transducer to the receiving circuitry, so it acts as a receiver for the second pulse. The process continues for successive pulses, with each transducer in the array (except the first and last ones) acting sometimes as a transducer and sometimes as a receiver. A signal processing unit 1214 then processes the signals, in much the same way as in the linear and circular moving systems, and an image reconstruction module 1216 creates the image. Using only one set of transmitting circuitry and one set of receiver circuitry, instead of having a separate set connected to each transducer in the array, makes the system much less expensive, and consume much less power, than a conventional ultrasound array of the same size, in which all of the transducers can transmit simultaneously, or receive simultaneously.

Alternatively, there is no acoustically transparent window 1204 between the transducer array and the surface of the body, but the transducer array is in direct contact with the body, for example using a gel to make good acoustic contact. This is quite practical in the case of the pseudo-motion system, because the transducers are not physically moving along the surface of the body, so there is no concern with drag, or damage to the body.

Pseudo-motion system is optionally 1 cm, 2 cm, 5 cm, 10 cm, or 20 cm in length, or a smaller, larger or intermediate length. If array 1202 is a two-dimensional array, to allow multiple slices to be imaged without moving the array, then it is optionally 1 cm, 2 cm, 5 cm, 10 cm, 20 cm, or a smaller, intermediate or larger length, in either dimension. Optionally these lengths are 10, 20, 50, 100 or 200 times the wavelength, in the body, or ultrasound of the average frequency used, or a smaller, intermediate, or greater number. Because the cost of the pseudo-motion array may be proportional to the number of elements, and hence proportional to the area of the array, the pseudo-motion system may be relatively more cost effective for imaging smaller regions, while the circular motion system may be relative more cost effective for imaging larger regions. In particular, the pseudo-motion system, which may have no moving parts and may not consume too much power, may be useful for continuously monitoring small regions of the body.

In some embodiments of the invention, there is only one row of transducers in array 1202, and the whole array is physically moved transversely to its length, in order to image other slices.

It should be understood that each of the “transducers” in array 1202 may itself be an array of transducer elements, arranged in a direction across the slice thickness, which are used for beam forming, i.e. to keep the transmitted beam confined largely to the slice they are imaging, and/or to limit the received signal largely to directions within the slice they are imaging.

Optionally the transducers in array 1202 are arranged substantially in a line, or their centers are. If some of the transducers are used only for transmitting, and some are used only for receiving, then optionally the transmitting transducers are substantially in a straight line, and the receiving transducers are substantially in a straight line, optionally parallel to the line of the transmitting transducers. Optionally, a planar imaging slice of finite thickness extends straight down into the body from the line of transducers, though generally with a dead region close to the transducers, and the transducers may only adjacent to the surface of the body, separated from it by an acoustically transparent plate, rather than directly on the surface of the body. If the transducers each comprise an array arranged across the thickness of the slice, then optionally the imaging region extends into the slice at an oblique angle, similar to what is shown in FIG. 6.

To show that pseudo-motion system 1200 will behave in approximately the same way as system 100 in which the transducers are actually moving, it should be noted that even in the case of continuous motion of the transducer, measurements of echoes have a discrete nature and are performed at time intervals of T, the pulse repetition interval. During this interval the transducer will move by Δx=Vₓ·T. Assume there is a localized scatterer in the imaging region at a depth R_{b}>>Δx. The change of distance to this point from two successive positions of the transducer, starting at time t_{b}, is

$$Δ R = \sqrt{{R}_{b}^{2} + Δ {x}^{2}} - {R}_{b} = \frac{Δ {x}^{2}}{2 {R}_{b}} - \frac{Δ {x}^{4}}{8 {R}_{b}^{3}} - … \approx \frac{Δ {x}^{2}}{2 {R}_{b}} = \frac{{( {V}_{x} \cdot T )}^{2}}{2 {R}_{b}} .$$

This change of distance will cause a change of phase of each spectral component f of the received signals relative to the appropriate spectral component f of the transmitted signals:

$$Δ ϕ = 2 π \frac{ 2 Δ R}{c} {F}_{c} = 4 π \frac{{( {V}_{x} \cdot T )}^{2}}{{R}_{b} \cdot c / {F}_{c}} = 4 π \frac{{V}_{x}^{2}}{{R}_{b} \cdot λ} {T}^{2} .$$

The phase shift of the nth pulse after time t_{b} will be

$${ϕ}_{n} = 4 π \frac{{V}_{x}^{2}}{{R}_{b} \cdot λ} {( n \cdot T )}^{2} = Δ {ϕ}_{0} \cdot {n}^{2} , \\ where$$

$$Δ {ϕ}_{0} = 4 π \frac{{( {V}_{x} \cdot T )}^{2}}{{R}_{b} \cdot λ}$$

is the phase shift of the first pulse after time t_{b}. This quadratic phase change of successive pulses means a linear frequency change, which is equivalent to the Doppler shift of the continuous motion system.

This result leads to the following concept for a static ultrasound system. A line of identical transducers, each of which can operate either as a transmitting or receiving transducer, can imitate the process of “movement” by sequentially switching the transmitting and receiving transducers in the line. The process can be described as follows: first transducer 1 transmits a pulse and transducer 2 receives the echo, after the pulse repetition interval, transducer 2 transmits a pulse and transducer 3 receives the echo. The process is repeated up to transducer number N−1 acting as a transmitter, and transducer N acting as a receiver for the echo. All received signals are collected and assembled to produce a matrix similar to that produced by system 100, with a real moving transducer. The effective “velocity” of the transducer in pseudo-movement system 1200 is Vₓ=Δx/T, where Δx is the separation between successive transmitting and receiving transducers, and T is the pulse repetition interval.

In the linear and circular moving systems, successive pulses should preferably be transmitted and received at spacing of no more than half a wavelength of the center frequency, which is the optimum spacing. This spacing avoids ambiguity in the phase change from one transmitting/receiving location to the next. For the same reason, transducer array 1202 preferably has transducers spaced at no more than half a wavelength, optionally at about half a wavelength. For this reason, it is potentially advantageous to have every transducer acting once as a transmitter and once as a receiver, so that the transmitting locations are spaced no more than half a wavelength apart, and the receiving location are spaced no more than half a wavelength apart.

Although it may be convenient to activate the transducers in the order in which they are arranged in the array, which would simulate the physical motion of a moving transducer, there is no need to do that, in order to reconstruct an image. The data on which the image is based consists of the received signal, including its phase relative to the transmitted pulse, for a pulse transmitted by each of the transmitting locations, and received by the corresponding receiving location (typically close to the transmitting location). The order in which the different locations are used, and even the timing between the pulses sent from the different locations, makes no difference, although in practice the phase differences between the transmitted pulse and received signal may be measured most easily by using a very stable local oscillator which provides an absolute time standard for transmitting and receiving of all the pulses. Sending the pulses as rapidly as possible, with just enough time between them to receive the echoes, has the advantage of reducing the time needed to obtain an image. But the transmitting/receiving locations could be activated in any order, as long as the signals are labeled to identify the transmitting and receiving locations of each pulse, and the data is then put in order of the transmitting and receiving locations, in order to apply the image reconstruction algorithms described in FIG. 3.

Optionally, the transmitting and receiving transducers are within 10 mm of each other for all pulses, or within 5 mm, or within 2 mm, or within 1 mm. Optionally, the transmitting and receiving transducers are a constant distance apart for all pulses.

### Medical Applications

The ultrasound imaging methods and systems described above provide some useful capabilities for medical ultrasound imaging, that are not available from prior art ultrasound imaging methods and systems. One advantage is the lateral resolution, which in the systems described here is, at least ideally, independent of depth in the imaging region, at least up to a depth about equal to the transducer path length, or array length in the case of a pseudo-motion system such as system 1200 in FIG. 12. In conventional ultrasound imaging, by contrast, lateral resolution is based on the angular width of the transmitted beam, which gets wider in proportion to depth, so the lateral resolution gets worse in proportion to depth. In conventional ultrasound imaging, the angular width of the beam is typically diffraction limited, based on the width of the transducer array generating the ultrasound. Producing a narrower beam, for a given size of transducer array, requires using higher frequency ultrasound. However, ultrasound at higher frequencies, above 2 MHz, has a penetration depth in body tissue that goes down rapidly with increasing frequency, about 1 db per cm per MHz, so it is still not possible to get very high lateral resolution at large depth. The lateral resolution measured by the inventors, in tests using a phantom with the properties of body fat, 0.5 mm at a depth of 10 cm, is beyond the capabilities of conventional ultrasound imaging. This was done using 2.5 MHz ultrasound. If 1.5 MHz ultrasound were used, which can penetrate to 30 cm, even in fat, it is expected that a lateral resolution of 0.8 mm or better could be achieved at depths of 20 or 30 cm, or even 40 cm, if the path length or transducer length were long enough. Such a system might be especially useful for high resolution ultrasound imaging of the abdomen in obese patients, which cannot be done with conventional ultrasound.

The systems described here can also have higher depth resolution than conventional ultrasound imaging systems, because they use pulses with high bandwidth times pulse length, B₀Tₚ>>1, which are less subject to distortion by the effects of dispersion, than imaging systems that use short pulses, with B₀Tₚ≈1. Conventional ultrasound imaging systems cannot use B₀Tₚ>>1, because they use very narrow focused beams, in order to get high lateral resolution. The inventors believe that B₀Tₚ=130 can be achieved at 1.5 MHz, and even higher B₀Tₚ should be achievable at higher frequencies.

High resolution abdominal imaging, particularly in obese patients, is one area where the present invention would offer an advantage in penetration depth and resolution, over conventional ultrasound imaging. To image the entire abdomen in such patients might require going to a depth of 40 cm or more. Using prior art imaging methods, such large imaging regions can only be imaged at resolutions of about 1 mm by CT and by MRI. But CT exposes patients to high levels of x-rays, and both CT and MRI require large expensive equipment, which cannot be used outside an institutional setting, and cannot be used for continuous monitoring. An ultrasound imaging system such as the ones described here would be relatively inexpensive and portable, so could be used at home or by emergency medical personnel, as well as for continuous monitoring of patients in a hospital. Moreover, because this type of ultrasound imaging has resolution similar to CT and MRI, it can be used in conjunction with CT and MRI to produce fused images which might better distinguish soft tissues than either CT or MRI by itself.

Another promising application for ultrasound imaging systems of the type described here, is in planning surgery, and for monitoring surgery in real time. It should be noted that not only is the resolution higher than in conventional ultrasound imaging, but the position information in the image is absolute, unlike conventional ultrasound imaging. Optionally, the absolute position information of a small object in the image is accurate to within 2 mm, or 1 mm, or 0.5 mm, or smaller, larger or intermediate values, at least down to a depth of 10 cm, or 20 cm, or 30 cm. Hence this type of ultrasound imaging can be used for planning surgery, and for monitoring surgery in real time, to avoid removing healthy tissue for example. While there exist x-ray imaging systems, and MRI systems, that can be used in real time during surgery, the x-ray systems expose patients to ionizing radiation, as well as the surgeon and other medical personnel to some extent. For this reason, such x-ray systems typically only create an image at infrequent intervals, for example once every 10 seconds, and creating a fully 3-D CT image during surgery would probably not be practical, since the x-ray exposure would be even greater, and the equipment would get in the way of the surgeon. Open access MRI systems for use during surgery also exist, but they generally have worse resolution than closed MRI systems. Furthermore, the expense of such MRI systems precludes their use in most surgery. An ultrasound system, however, does not expose anyone to ionizing radiation, and is inexpensive and unobtrusive enough to use routinely.

It is expected that during the life of a patent maturing from this application many relevant matched filtering methods will be developed and the scope of the term matched filter is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and computational support in the following examples.

### EXAMPLES

Reference is now made to the following examples, which together with the above descriptions illustrate some embodiments of the invention in a non limiting fashion.

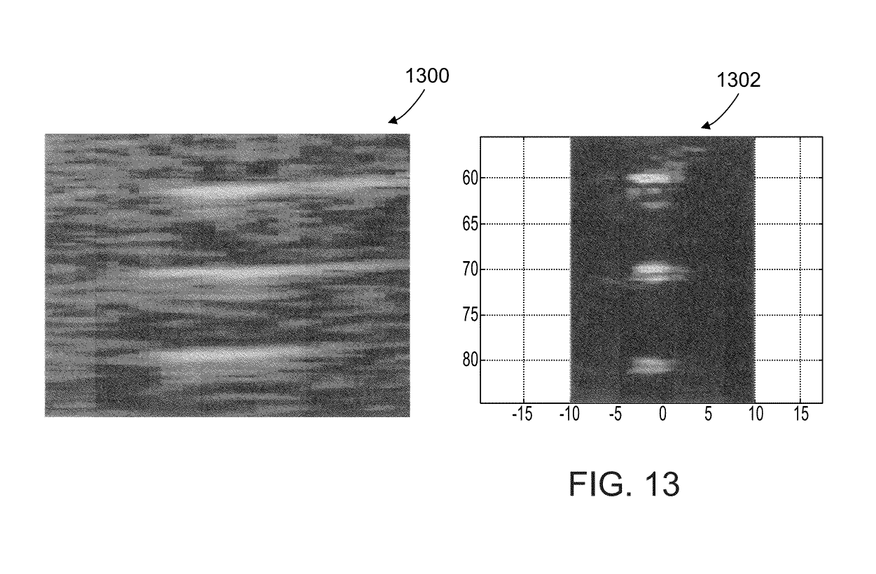

FIG. 13 shows data from a test made with a phantom, comparing the resolution of a conventional (B-Mode) ultrasound scan, and an ultrasound scan using a pseudo-motion system such as described in FIG. 12, with 64 transducers in an array that is 15 mm long. In both cases, a short pulse, sinc(ωt), was used, rather than a linearly frequency modulated pulse, at 2.5 MHz. Plot 1300 shows a portion of the conventional B-Mode ultrasound scan, showing 3 point reflectors, vertically arranged at depths of 6, 7 and 8 cm. The depth resolution is about 1 mm, and the lateral resolution is about 6 or 7 mm Plot 1302 shows a portion of an ultrasound scan using the pseudo-motion system, showing the same part of the phantom, on the same scale. The depth resolution is about the same as in the B-Mode scan, 1 mm, but the lateral resolution is much better than in the B-Mode scan, about 1.5 mm. Furthermore, in the pseudo-motion system scan, the lateral resolution is not noticeably worse at 8 cm depth than at 6 cm depth, while in the B-Mode scan it is.

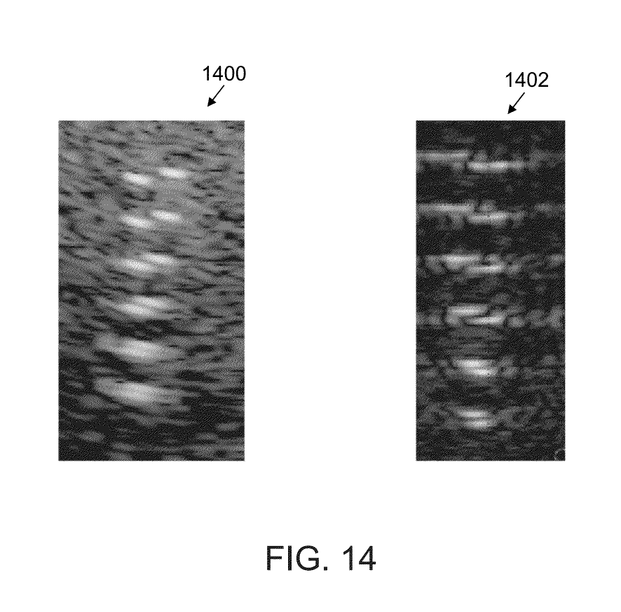

FIG. 14 shows test results on lateral resolution, using General Purpose Urethane Ultrasound Phantom Model 042, sold by CIRS, Inc. The material in this phantom has ultrasound absorption properties similar to fat. This phantom includes six pairs of essentially point reflectors at depths of 7.2, 7.8, 8.4, 9.0, 9.6, and 10.2 cm, each pair separately laterally by a different distance, respectively 5 mm, 4 mm, 3 mm, 2 mm, 1 mm, and 0.5 mm. Each pair is also separated by 1 mm in depth. FIG. 14 shows an image 1400 of these pairs of reflectors made by conventional B-Mode ultrasound imaging, and an image 1402 of the same reflectors using a pseudo-motion system similar to that shown in FIG. 12, with a 64-element transducer array that is 15 mm long, both images using 2.5 MHz ultrasound. Note that in image 1402 even for the deepest pair of reflectors, at a depth of 10.2 cm, it is possible to see that one of the reflectors is laterally displaced from the other one, showing that the system makes it possible to determine differences in the lateral position of a point reflector of only 0.5 mm. In image 1400, by contrast, it is barely possible to see the difference in lateral position for the fourth pair down, with a separation of 2 mm, at a depth of 9.0 cm. This shows that the lateral resolution, at a depth of 10 cm, is at least 4 times better for the pseudo-motion system, than for a B-Mode system. The improvement in resolution is expected to be even greater at greater depths, since the lateral resolution of the pseudo-motion system is relatively independent of depth, while the lateral resolution of the B-Mode system gets worse in proportion to depth.

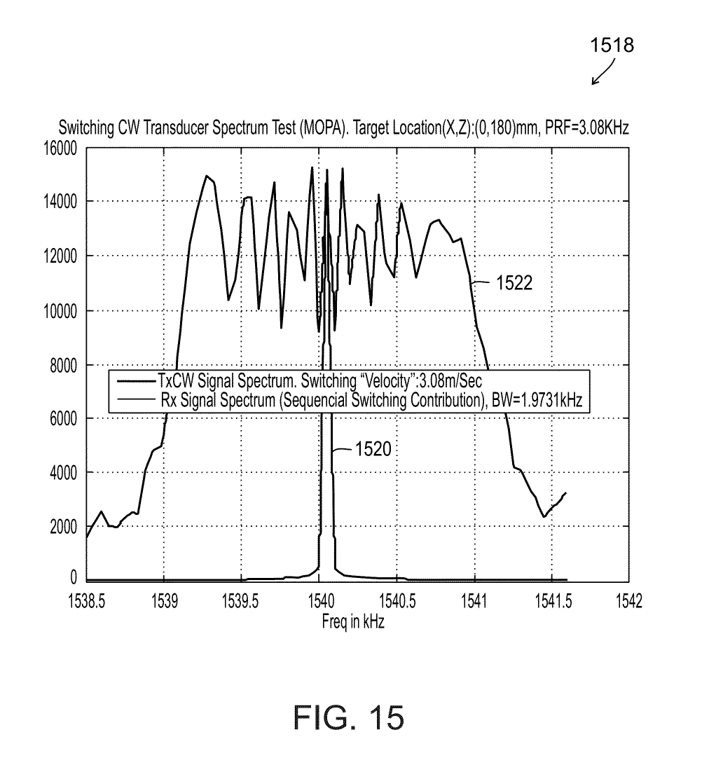

FIG. 15 shows a plot 1518 of the frequency spectrum of the transmitted and received pulses, in a simulation of pseudo-motion system 1200, in order to illustrate the “pseudo-Doppler shift” caused by the pseudo-motion of the transducer. The simulated system has an array of 64 transducers, each one transmitting one pulse at regular time intervals in the order that they are arranged in the array, and the array is 25 mm wide, using 1.54 MHz ultrasound. Curve 1520 shows the frequency spectrum of the transmitted pulses, which is very narrow because the frequency of the transmitted pulses is coherent over the entire set of pulses that is transmitted. Curve 1522 shows the frequency spectrum of the received pulses, for a single localized scatterer at a depth of 180 mm. The phase shifts in the received pulses for the different transducers, which are approximately a quadratic function of the position of the transducer and of the timing of the transmitted pulse, introduce a broadening of the frequency spectrum of the received signal data, when the received signals from all the pulses are treated as a single time sequence.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of producing an ultrasound image of an imaging region of a body, the image comprising pixels, the method comprising:
a) transmitting time-varying ultrasound into the imaging region, over a time interval, from a surface of the body, the transmitted ultrasound simultaneously having an angular spread in the imaging region corresponding to a plurality of the pixels of the image; and
b) receiving echoes of the transmitted ultrasound, and recording received signals of the echoes;
wherein one or both of the transmitting and the receiving is done at a different location during each of a plurality of different sub-intervals of the time interval; and
c) combining the received signals at the different sub-intervals of the time interval based on said time varying, according to expected ultrasound propagation times to scatterers localized at different pixels, to find image densities of scatterers at the pixels.

2. A method according to claim 1, wherein combining the received signals at the different sub-intervals comprises using a dependence of a phase of the received signals on sub-interval, due to a change in the location of the transmitting, the receiving, or both, in the different sub-intervals, resulting in a change in distance from the location of transmitting to a scatterer in the imaging region to the location of receiving, to distinguish echoes from different scatterer locations.

3. (canceled)

4. A method according to claim 2, wherein combining the received signals at the different sub-intervals comprises finding a spectrum of at least the dependence of phase of the received signals on sub-interval, the method also comprising using the spectrum to find a density of ultrasound scatterers as a function of azimuthal direction from the transducers, and range.

5. (canceled)

6. A method according to claim 2, wherein combining the received signals at the different sub-intervals comprises matching the dependence of the phase of the received signals on sub-interval to a dependence of phase of the received signals on sub-interval that would be seen for scatterers located at different depths and lateral positions.

7. (canceled)

8. A method according to claim 1, wherein the time-varying ultrasound within each sub-interval is frequency modulated, and combining the received signals at the different sub-intervals comprises:
a) using a phase dependence of the received signal within sub-intervals to find a distribution of ranges of the echoes for the different sub-intervals; and
b) using phase differences between different sub-intervals, to find a distribution of azimuthal directions of the echoes for different ranges;
wherein the phase changes within a sub-interval are at least 30 times as fast as the phase changes between sub-intervals.

9. A method according to claim 1, wherein combining the received signals at the different sub-intervals comprises using differences in both amplitude and phase of the received signals at the different sub-intervals.

10. (canceled)

11. A method according to claim 1, wherein the image densities at the pixels have a resolution in a direction lateral to the depth, that decreases by less than 30%, over at least one portion of the imaging region in which the depth increases by a factor of 2 or more.

12. A method according to claim 1, wherein the image densities of the pixels have a resolution of better than 2 mm in a lateral direction, at a depth of at least 20 cm.

13. (canceled)

14. A method according to claim 12, wherein the depth of at least 20 cm includes at least 10 cm of fat.

15.-40. (canceled)

41. A method according to claim 1, wherein the imaging region is comprised in a slice extending into the body from said surface, the transmitting and receiving locations being on or adjacent to a portion of the surface that the slice extends from, the portion being narrower in a direction across the slice than in a direction along the slice.

42.-50. (canceled)

51. A method according to claim 1, wherein a lateral resolution of the image is better than one wavelength of an average frequency of the transmitted ultrasound, down to a depth of at least 20 such wavelengths beneath the surface.

52. (canceled)

53. A system for calculating density values of scatterers localized at pixels of an ultrasound image of an imaging region of a body, comprising:
a) one or more transmitting transducers adapted to transmit time-varying ultrasound waves into the imaging region, when placed on or adjacent to a surface of the body at any of a plurality of different transmitting locations;
b) one or more receiving transducers, the same as or different from the transmitting transducers, adapted to receive an echo of the transmitted ultrasound waves from scatterers in the imaging region, when placed on or adjacent to the surface at any of a plurality of different receiving locations, and to generate a received signal thereof;
c) a location changing device adapted to change the transmitting location, or adapted to change the receiving location, or adapted to change both; and
d) a controller adapted to calculate the image density value of scatterers localized at different pixels, by combining the received signals according to expected ultrasound propagation times to and from the pixels, for a plurality of different transmitting locations, a plurality of different receiving locations, or both.

54. A system according to claim 53, wherein the location changing device is adapted to move a transmitting transducer, a receiving transducer, or both, from one of the locations to another.

55. A system according to claim 54, comprising a movement sensor that senses the location as a function of time of at least one transducer that is moved by the location changing device, and generates data thereof, wherein the controller is adapted to use said data to adjust the expected ultrasound propagation times.

56. A system according to claim 54, wherein the location changing device is adapted to move the transducer substantially in a straight line or geodesic along or adjacent to said surface of the body.

57. A system according to claim 56, wherein the location changing device comprises:
a) a rigid acoustically transparent plate, a lower surface of which is adapted to remain in good acoustic contact with the surface of the body, over a range of the locations; and
b) a motor adapted to move the transmitting transducer, receiving transducer, or both, along an upper surface of the plate, opposite the lower surface, over the range of the locations, while the transducer remains facing the plate, in direct or indirect contact with the upper surface.

58. (canceled)

59. A system according to claim 54, wherein the location changing device comprises a motor adapted to move the transducer substantially in a circular arc.

60. A system according to claim 59, wherein the location changing device comprises a rigid circular rotating portion on which the transducer is mounted, and a motor that causes the rotating portion to rotate, and the system also comprises a stationary portion that remains fixed when the rotating portion rotates.

61.-64. (canceled)

65. A system according to claim 53, wherein the one or more transmitting transducers comprise an array of transmitting transducers, the one or more receiving transducers comprise an array of receiving transducers, or both, and the location changing device comprises switching circuitry that switches which transducer in the array of transmitting transducers is used for transmitting, or which transducer in the array of receiving transducers is used for receiving, or both.

66.-71. (canceled)

72. A method of producing an ultrasound image of an imaging region of a body, the image comprising pixels, the method comprising:
a) transmitting time-varying ultrasound into the imaging region, over a time interval, from a surface of the body, the transmitted ultrasound simultaneously having an angular spread in the imaging region corresponding to a plurality of the pixels of the image; and
b) receiving echoes of the transmitted ultrasound, and recording received signals of the echoes;
wherein one or both of the transmitting and the receiving is done at a plurality of different locations; and
c) combining the received signals based on said time varying, according to expected ultrasound propagation times to scatterers localized at different pixels, to find image densities of scatterers at the pixels.

73. A system according to claim 53, wherein the one or more transmitting transducers are adapted to transmit time-varying ultrasound waves into the imaging region with an angular spread of at least 20 degrees.
